(19) United States
(12) Patent Application Publication
(10) Pub. No.: US 2007/0099197 A1
(43) Pub. Date: May 3, 2007

(54) Methods of prognosis of prostate cancer

(76) Inventors: Daniel E.H. Afar, Fremont, CA (US); Susan M. Henshall, New York, NY (US); Jordan B. Hiller, San Mateo, CA (US); David H. Mack, Menlo Park, CA (US); Robert L. Sutherland, Lindfield (AU)

Correspondence Address: FENWICK & WEST LLP, SILICON VALLEY CENTER, 801 CALIFORNIA STREET, MOUNTAIN VIEW, CA, 94041, US

(21) Appl. No.: 11/365,745

(22) Filed: Feb. 28, 2006

Related U.S. Application Data
(63) Continuation of application No. 10/603,505, filed on Jun. 24, 2003, now abandoned.
(60) Provisional application No. 60/391,309, filed on Jun. 24, 2002.

(51) Int. Cl. C12Q 1/68; C12P 19/34

(52) U.S. Cl. 435/6; 435/91.2

(57) ABSTRACT

The present invention applies classical survival analysis to genome-wide gene expression profiles of prostate cancers and preoperative prostate-specific antigen levels from prostate cancer patient, to identify prognostic markers of disease relapse that provide additional predictive value relative to prostate-specific antigen concentration. The present invention provides a method of determining prognosis of prostate cancer and predicting prostate cancer outcome of a patient. The method comprises the steps of first establishing the threshold value of at least one prognostic gene of prostate cancer. Then, the amount of the prognostic gene from a prostate tissue of a prostate cancer patient is determined. The amount of the prognostic gene present in that patient is compared with the established threshold value of the prognostic gene, whereby the prognostic outcome of the patient is determined.

This application is a continuation of pending U.S. patent application Ser. No. 10/603,505, filed Jun. 24, 2003, which claims the benefit of provisional application 60/391,309, filed Jun. 24, 2002, which is incorporated herein in its entirety.

### FIELD OF THE INVENTION

The invention relates to the identification of nucleic acid and protein expression profiles and nucleic acids, products, and antibodies thereto that are outcome prognostic in prostate cancer.

### BACKGROUND OF THE INVENTION

Prostate cancer will account for an estimated 30% (189,000) of new cancer cases in men in the United States in 2002 (1). Many of these newly diagnosed cases are a result of the extensive use of prostate-specific antigen (PSA) screening and the subsequent diagnosis of prostate cancer at an early stage and age. However, despite the introduction of PSA screening the mortality from prostate cancer has remained relatively constant. The implications of this are that: (1) there are a large group of men diagnosed with prostate cancer for whom radical treatment is probably unnecessary and who will die with their prostate cancer rather than from it; and (2) there are a group of men for whom early detection offers the possibility of cure that may be denied by delay. Consequently, identifying these groups of men at the time of diagnosis is critical to the optimal management of prostate cancer.

While the benefits of PSA screening are widely debated, this serum marker remains one of only a small number of preoperative parameters of prognostic utility. In order to enhance the predictive value of individual parameters with outcomes, nomograms have been developed that incorporate parameters that are measured routinely in clinical practice to predict the probability of PSA relapse free survival of individual patients both prior to and following therapy (2-6). Models such as these currently form the basis of routine clinical decision-making, but such classification systems cannot explore differences in outcomes observed between cancers with similar histopathological features. Hence, there remains a critical need for increased accuracy in the subcategorization of prostate cancers to identify those with an aggressive phenotype.

There are a considerable number of publications assessing the ability of biomarkers to predict an earlier time to relapse of prostate cancer following radical prostatectomy (reviewed in ref. (17)). Despite these data, there remain no molecular markers of routine clinical utility which differentiate localized prostate cancers with an aggressive phenotype, and clinicians still rely on conventional preoperative and postoperative prognostic indicators such as pretreatment PSA levels, pathological stage and Gleason grade in routine decision-making. This most likely reflects the fact that studies that have correlated differences in gene expression with patient outcome have assessed candidate genes with limited predictive power that provide no additional prognostic information above the conventional variables. This accentuates the need to discover novel genes with strong predictive ability.

One approach is to define patterns of gene expression that correlate with disease phenotype and patient outcome. Here, we undertook a systematic search for novel biomarkers of prostate cancer prognosis by outcome-based analyses of transcript profiles.

### SUMMARY OF INVENTION

The present invention evaluates gene expression profile and identifies prognostic genes of prostate cancers. The present invention provides a method of determining prognosis of prostate cancer and predicting prostate cancer outcome of a patient. The method comprises the steps of first establishing the threshold value of at least one prognostic gene of prostate cancer. Then, the amount of the prognostic gene from a prostate tissue of a patient inflicted of prostate cancer is determined. The amount of the prognostic gene present in that patient is compared with the established threshold value of the prognostic gene, whereby the prognostic outcome of the patient is determined.

In certain embodiments, the amount of the prognostic gene is determined by the quantitation of a transcript encoding the sequence of the prognostic gene; or a polypeptide encoded by the transcript. The quantitation of the transcript can be based on hybridization to the transcript. The quantitation of the polypeptide can be based on antibody detection. The method optionally comprises a step of amplifying nucleic acids from the tissue sample before the evaluating. In some embodiments, the evaluating is of a plurality of sequences. The method may further comprises determining prostate-specific antigen (PSA) level. The prognosis contributes to selection of a therapeutic strategy.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1 shows cluster analysis of prostate cancer samples from 72 patients treated for localised prostate cancer by RP. Each column represents a single RP specimen and each row represents one of the 264 genes which demonstrated a strong association with PSA relapse in our model. The dendogram at the top shows the degree to which each prostate cancer is related to the others with respect to gene expression. The 17 patients known to have experienced a PSA relapse are indicated by an “R”. The relative level of expression is indicated by the color scale at the bottom and is indicative of the normalized average intensity units of fluorescence signal detected by microarray analysis.

FIG. 2A shows the expression of trp-p8 mRNA detected by oligonucleotide microarray in prostate cancer samples and in normal body tissues. Samples are: prostate cancer 1-74, adrenal glands 75-77, aorta 78-80, artery 81-83, bladder 84-86, bone marrow 87-89, colonic epithelium 90-92, cerebral cortex 93-95, colon 96-98, colonic muscle 99-101, esophagus 102-104, heart 105-107, kidney 108-110, liver 111-113, lung 114-116, lymph node 117-119, muscle 120-122, oral mucosa 123-125, pharyngeal mucosa 126-128, pancreas 129-131, parathyroid glands 132-133, pituitary 134-136, prostate 137-143, retina 144-146, skin 147-149, small intestine 150-152, spleen 153-155, stomach 156-158, trachea 159-161, tongue 162-164, ureter 165-167, vagus nerve 168-170, vein 171-174.

FIG. 2B shows the expression of trp-p8 mRNA, and FIG. 2C shows the PSA mRNA; both detected by oligonucleotide microarray in LuCaP-35 tumors at days 0 to 100 post castration. The expression level of trp-p8 and PSA is shown as normalized average intensity units (Y-axis) of fluorescence signal detected by microarray analysis.

units (Y-axis) of fluorescence signal detected by microarray analysis.

FIG. 3 shows the Trp-p8 mRNA expression detected by in situ hybridization in radical prostatectomy cases treated with or without neoadjuvant hormone therapy prior to surgery.

FIG. 3A: A prostate cancer from a patient treated with RP only showing positive trp-p8 mRNA expression in malignant prostate epithelium. FIG. 3B: A prostate cancer from a patient treated with RP and NHT showing positive trp-p8 mRNA expression. FIG. 3C: A prostate cancer from a patient treated with RP only with no detectable trp-p8 mRNA expression in the malignant epithelium, and Figure D: A prostate cancer from a patient treated with preoperative NHT with no evidence of trp-p8 expression.

FIG. 4A shows the trp-p8 protein sequence. FIG. 4B shows the trp-p8 mRNA sequence.

### DETAILED DESCRIPTION OF INVENTION

Current models of prostate cancer classification are poor at distinguishing between tumors that have similar histopathological features but vary in clinical course and outcome. In the present invention, we have applied classical survival analysis to genome-wide gene expression profiles of prostate cancers and preoperative prostate-specific antigen levels from each patient, to identify prognostic markers of disease relapse that provide additional predictive value relative to prostate-specific antigen concentration. The present invention demonstrates that multivariable survival analysis can be applied to gene expression profiles of prostate cancers with censored follow-up data and used to identify molecular markers of prostate cancer relapse with strong predictive power and relevance to the etiology of this disease.

Prostate Cancer and Treatments

Prostate cancer is found mainly in older men. Prostate cancer is the most commonly diagnosed internal malignancy and second most common cause of cancer death in men in the U.S., resulting in approximately 40,000 deaths each year. Landis et al. (1998) CA Cancer J. Clin. 48:6-29; and Greenlee, et al. (2000) CA Cancer J. Clin 50:7-13. The incidence of prostate cancer has been increasing rapidly over the past 20 years in many parts of the world. Nakata, et al. (2000) Int. J. Urol. 7:254-257; and Majeed, et al. (2000) BJU Int. 85:1058-1062. It develops as the result of a pathologic transformation of normal prostate cells. In tumorigenesis, the cancer cell undergoes initiation, proliferation, and loss of contact inhibition, culminating in invasion of surrounding tissue and, ultimately, metastasis.

Prostate cancer is a disease in which malignant (cancer) cells form in the tissues of the prostate. The prostate is a gland in the male reproductive system located just below the bladder (the organ that collects and empties urine) and in front of the rectum (the lower part of the intestine). It is about the size of a walnut and surrounds part of the urethra (the tube that empties urine from the bladder). The prostate gland produces fluid that makes up part of the semen. See generally, Boyle, et al. (2002) Textbook of Prostate Cancer Isis Medical Media, ISBN: 1901865304; Kantoff (ed. 2002) Prostate Cancer: Principles and Practice Lippincott, ISBN: 0781720060; Carroll (2001) Prostate Cancer Decker, ISBN: 1550091301; Belldegrun, et al. (2000) New Perspectives in Prostate Cancer Isis Medical Media, ISBN: 1901865568; Lepor (1999) Prostatic Diseases Saunders, ISBN: 072167416X; Petrovich, et al. (eds. 1996) Carcinoma of the Prostate: Innovations in Management, Springer Verlag, ISBN: 3540587497; and standard prostate cancer medical texts.

Four types of standard treatment are used for prostate cancer: watchful waiting, surgery, radiation therapy, or hormone ablation therapy. See, e.g., the National Cancer Institute (NCI) description of prostate cancer, www.cancer.gov.

Watchful waiting is closely monitoring a patient's condition but withholding treatment until symptoms appear or change. This is usually used in older men with other medical problems and early stage disease.

Surgery is usually offered to prostate cancer patients in good health who are younger than 70 years old. Main surgery options are pelvic lymphadenectomy, radical protatectomy, perineal prostatectomy, and transurethral resection of the prostate.

Pelvic lymphadenectomy is a surgical procedure to take out lymph nodes in the pelvis to see if they contain cancer. If the lymph nodes contain cancer, the doctor will not remove the prostate and may recommend other treatment. Radical prostatectomy (RP) is surgery to remove the entire prostate. Radical prostatectomy is done only if tests show the cancer has not spread outside the prostate. The two types of radical prostatectomy are retropubic prostatectomy, which removes the prostate through an incision made in the abdominal wall, and removal of surrounding lymph nodes (lymphadenectomy) can be done at the same time; and perineal prostatectomy, which is surgery to remove the prostate through an incision made between the scrotum and the anus, and if surrounding lymph nodes are to be removed, this is usually done through a separate incision. Transurethral resection of the prostate is a surgical procedure to remove tissue from the prostate using an instrument inserted through the urethra. This operation is sometimes done to relieve symptoms caused by the tumor before other treatment is given. Transurethral resection of the prostate may also be done in men who cannot have a radical prostatectomy because of age or illness.

Impotence and leakage of urine from the bladder or stool from the rectum may occur in men treated with surgery. In some cases, doctors can use a technique known as nerve-sparing surgery. This type of surgery may save the nerves that control erection. However, men with large tumors or tumors that are very close to the nerves may not be able to have this surgery.

Radiation therapy is the use of x-rays or other types of radiation to kill cancer cells and shrink tumors. Radiation therapy may use external radiation (using a machine outside the body) or internal radiation. Internal radiation involves putting radioisotopes (materials that produce radiation) through thin plastic tubes into the area where cancer cells are found. Prostate cancer is treated with external and internal (implant) radiation. Radiation therapy may be used alone or in addition to surgery. Impotence and urinary problems may occur in men treated with radiation therapy.

Hormone therapy is the fourth of the standard treatments. Hormones are chemicals produced by glands in the body and circulated in the bloodstream. Hormone therapy is the use of hormones to stop cancer cells from growing. Male hormones (especially testosterone) can help prostate cancer grow. To stop the cancer from growing, female hormones or drugs that decrease production of male hormones may be given. Hormone therapy used in the treatment of prostate cancer may include the following: estrogens (hormones that promote female sex characteristics) can prevent the testicles from producing testosterone, however, estrogens are seldom used today in the treatment of prostate cancer because of the risk of serious side effects; luteinizing hormone-releasing hormone agonists also can prevent the testicles from producing testosterone, e.g., leuprolide, goserelin, and buserelin; antiandrogens can block the action of androgens (hormones that promote male sex characteristics), two examples are flutamide and bicalutamide; drugs that can prevent the adrenal glands from making androgens include ketoconazole and aminoglutethimide; and orchiectomy is surgery to remove the testicles, the main source of male hormones, to decrease hormone production. Hot flashes, impaired sexual function, and loss of desire for sex may occur in men treated with hormone therapy.

Deaths from prostate cancer are typically a result of metastasis of a prostate tumor. Therefore, early detection of the development of prostate cancer is critical in reducing mortality from this disease. Measuring levels of prostate-specific antigen (PSA) has become a very common method for early detection and screening, and may have contributed to the slight decrease in the mortality rate from prostate cancer in recent years. Nowroozi, et al. (1998) Cancer Control 5:522-531. However, many cases are not diagnosed until the disease has progressed to an advanced stage.

Prognosis, Outcome

Prognosis is typically recognized as a forecast of the probable course and outcome of a disease. See Dorland's Medical Dictionary. As such, it involves inputs of both statistical probability, requiring numbers of samples, and outcome data. Herein, outcome data is utilized in the form of prostate cancer recurrence after RP. A patient population of many dozens is included, providing statistical power.

The ability to determine which cases of prostate cancer will respond to treatment, and to which type of treatment, would be useful in appropriate allocation of treatment resources. As indicated above, the various standard therapies have significantly different risks and potential side effects. Accurate prognosis would also minimize application of treatment regimens which have low likelihood of success. Such also could avoid delay of the application of alternative treatments which may have higher likelihoods of success for a particular presented case. Thus, the ability to evaluate individual prostate cases for markers which subset into responsive and non-responsive groups for particular treatments are very useful.

Current models of prostate cancer classification are poor at distinguishing between tumors that have similar histopathological features but vary in clinical course and outcome. Kattan, et al. (1998) J. Nat'l Cancer Inst. 90:766-771; and Kattan, et al. (1999) J. Clin. Oncol. 17:1499-1507. Identification of novel prognostic molecular markers is a priority if radical treatment is to be offered on a more selective basis to those prostate cancer patients with clinically significant disease. A novel strategy is described to discover molecular markers for prostate cancer prognosis by assessing genome-wide gene expression in many localized prostate cancers and modeling these data based on each patient's known clinical outcome and preoperative serum prostate-specific antigen concentration. The study herein is directed to molecularly define different forms of prostate cancer which can translate directly into prognosis. And such prognosis allows for application of a treatment regimen having a greater statistical likelihood of cost effective treatments and minimization of negative side effects from the different treatment options.

Prostate cancer biopsy samples were collected and analyzed for gene expression across most genes of the human genome. Among genes detected at appropriate levels, correlations with outcome data were evaluated. Genes whose expression levels correlated with statistical significance to outcome data were identified.

This approach identified about 270 genes that demonstrated a strong association (P<0.01) with disease outcome, e.g., prostate cancer relapse, and were superior in their predictive ability relative to prostate-specific antigen levels, one of the standard markers. One of these genes, the putative calcium channel protein trp-p8, is androgen-regulated and loss of trp-p8 appears to be associated with aggressive disease. The findings provide a method of survival analysis of gene expression profiles of cancers with censored follow-up data and identify novel molecular markers of prostate cancer progression with strong predictive power that may be used to select prostate cancers with an aggressive phenotype.

Thus, the invention herein provides statistical correlations of marker expression in appropriate samples with disease outcome.

Survival Analysis

The present invention provides the application of classical multivariable survival analysis to a prostate cancer microarray data set incorporating the expression profiles of over 46,000 genes, to identify markers of disease outcome. This technique provides several significant advances over previous methods of analyses that have been used to discover markers of disease outcome from microarray data. In contrast to previously described statistical methods that rely on the classification of tumors based on known outcome (18) or known classifiers of patient outcome (eg. estrogen receptor status) (19, 20), this technique provides for censored data. This enables these analyses to proceed prior to the occurrence of all events, in this case, PSA relapse. Moreover, this survival analysis incorporates the time taken to PSA relapse and may also include covariates (eg. preoperative serum PSA levels) in order to identify genes that provide additional predictive value above conventional markers of outcome. The statistical analyses described herein have also incorporated a stringent method of estimating the pFDR that was recently described (10). This method is designed specifically for the analysis of microarray data where general dependence between hypotheses or “clumpy dependence” exists, where 50 or more genes interact in common pathways to produce some overall process (10). However, this is the first instance that it has been applied to microarray data from a survival analysis.

A recently published analysis to discover new markers of prostate cancer outcome utilized microarray analyses of prostate cancers to classify small groups of tumors where the recurrence status was known (21). That study found that no single gene was statistically associated with recurrence at P<0.05 and instead adopted a 5-gene model that most commonly included chromogranin A and inositol triphosphate receptor 3 (IP3R). The significant differences between our study and these previously published data are (1) our adoption of a Cox proportional hazards model, and (2) our observation that 277 individual genes were predictive for prostate cancer relapse, none of which overlapped with the genes in the 5-gene model identified by Singh et al. (2002). There are two prevailing explanations for the latter discrepancy. Firstly, the number of genes interrogated by oligonucleotide microarrays in our study was 4-fold greater; trp-p8 is an example of a gene which was not present in the oligonucleotide array used in the previous study. As a result, the genes identified by Singh et al. (2002), were associated with P values of less significance than those presented in Tables 1 and 2. Secondly, by utilizing a statistical method that applies to censored data, we were able to take into account the varying times to prostate cancer relapse in this model. Therefore, we were able to use our full data set in the analysis, rather than restricting the analysis to those patients with a specified length of follow-up. The larger data set and concomitant increase in statistical power may also contribute to our results differing from those of Singh et al.

The TRP channels are made of subunits with six membrane-spanning domains with both carboxy and amino termini located intracellularly that probably form into tetramers to form non-selective cationic channels, which allow for the influx of calcium ions into the cell. Trp-p8 or TRPM8 is a member of the TRPM subfamily of TRP ion channels that have potential roles in Ca²⁺-dependent signaling, control of cell cycle proliferation, cell division and cell migration. Ligand binding to some membrane receptors initiates a sequence of events that lead to the activation of phospholipase C, generating inositol-1,4,5-triphosphate which opens the intracellular ion channel IP3R and liberates Ca²⁺ from the endoplasmic reticulum. Activation of the TRP channels accompanies this chain of events, allowing the influx of calcium ions into the cells, although their activation is not necessarily directly linked to Ca²⁺ depletion from internal stores (22). Calnexin, which is also identified in this analysis as a marker of potential prognostic utility (P=0.004), is believed to be a key chaperone involved in the folding, assembly and oligomerization of newly synthesised IP3R receptors (24). Thus, our study implicates an important role for the phosphatidylinositol signal transduction.

Our observation that loss of trp-p8 is associated with a poor prognosis is also reminiscent of the prognostic role of another of the TRPM subfamily, TRPM1 or melastatin, in melanoma. Downregulation of melastatin mRNA in primary cutaneous melanoma is a prognostic marker for metastasis in patients with localized melanoma and is independent of conventional clinicopathological predictors of metastases (25). Recent studies showed that the rat (26) and mouse (27) orthologues of trp-p8 are functional calcium channels that respond to cold stimuli. Although cold is unlikely to be the natural stimulus for trp-p8 in the prostate, the implication that the human trp-p8 protein may be a functional Ca²⁺ channel suggests a role in the regulation of intracellular Ca²⁺ levels with possible effects on cell motility, cell proliferation and resistance to apoptotic stimuli.

In summary, our analyses have identified a group of genes that strongly correlate with prostate cancer relapse and contribute unique information to relapse prediction above preoperative PSA.

Prognosis Determination

One application of the survival analysis results is to generate a prognostic test for prostate cancer. First, we use TAQMAN® analysis to determine the absolute levels of prognostic genes in 75-150 or more prostate cancer patients. Then we correlate the absolute levels of the prognostic genes with patient outcome by a statistical analysis and determine threshold levels of prognostic genes; from which we establish a profile of the threshold level of each prognostic gene associated with a good outcome. For determining the prognosis of a prostate cancer patient, the absolute level of one or more prognostic genes of this patient is determined. Then the absolute level of one or more prognostic genes of this patient is compared with the above established threshold values. Absolute level higher (or lower depending on the prognostic gene) than the threshold values indicates good outcome.

The normalized quantitative level of absolute gene expression of a prognostic gene, from which outcome is predicted, is determined first. Quantitative polymerase chain reaction (PCR)-based methods can be applied. RT-PCR (reverse transcriptase PCR) primers are designed for selected prognostic genes, in order to perform a TaqMan® analysis.

TAQMAN® analysis is a real-time quantitative PCR, which is a powerful method used for gene expression analysis, genotyping, pathogen detection/quantitation, mutation screening and DNA quantitation. See, e.g., Bartlett (2003) PCR Protocols (2^{d} ed.) Humana Press; and O'Connell (2002) RT-PCR Protocols, Humana Press. The technology uses, e.g., an ABI Prism instrument (TAQMAN®) to detect accumulation of PCR products continuously during the PCR process thus allowing easy and accurate quantitation in the early exponential phase of PCR. The basis for PCR quantitation in the ABI instrument is to continuously measure PCR product accumulation using a dual-labeled flourogenic oligonucleotide probe called a TAQMAN® probe. This probe is composed of a short (ca. 20-25 bases) oligodeoxynucleotide that is labeled with two different flourescent dyes. On the 5′ terminus is a reporter dye and on the 3′ terminus is a quenching dye. This oligonucleotide probe sequence is homologous to an internal target sequence present in the PCR amplicon. When the probe is intact, energy transfer occurs between the two flourophors and emission from the reporter is quenched by the quencher. During the extension phase of PCR, the probe is cleaved by 5′ nuclease activity of Taq polymerase thereby releasing the reporter from the oligonucleotide-quencher and producing an increase in reporter emission intensity. The laser light source excites each well and a CCD camera measures the fluorescence spectrum and intensity from each well to generate real-time data during PCR amplification. The ABI Prism software examines the fluorescence intensity of reporter and quencher dyes and calculates the increase in normalized reporter emission intensity over the course of the amplification. The results are then plotted versus time, represented by cycle number, to produce a continuous measure of PCR amplification. To provide precise quantification of initial target in each PCR reaction, the amplification plot is examined at a point during the early log phase of product accumulation. This is accomplished by assigning a fluorescence threshold above background and determining the time point at which each sample's amplification plot reaches the threshold (defined as the threshold cycle number or CT). Differences in threshold cycle number are used to quantify the relative amount of PCR target contained within each tube as described previously.

The TAQMAN® primers are designed within the open-reading frame of the prognostic gene of interest so that the amplicon averages 80 bp. Prostate tissue samples from 70-150 or more prostate cancer patients with known histories are collected and RNA is extracted from these samples using standard methods. TAQMAN® analysis is performed on these samples for the appropriate genes. Using the TAQMAN® analysis, the normalized absolute levels of the prognostic genes are then correlated with patient outcome. Using statistical analyses the threshold level of gene expression, which predicts outcome, is then determined. Subsequent patient samples can then be analyzed for potential of relapse and the physician can better define the patient treatment based on whether the patient is predicted to relapse. Subsetting of the data into various outcomes is achieved through statistical analyses. (Snedecor and Cochran (1994) Statistical Methods (8^{th} ed.) Iowa State University Press; and Duda, et al. (2001) Pattern Classification (2^{d} ed.) Wiley and Sons.)

Genes, Markers, Kits

The present study provides specific identification of multiple genes whose expression levels in biological samples will serve as markers to evaluate prostate cancer cases. These markers have been selected for statistical correlation to disease outcome data on a large number of prostate cancer patients.

The expression levels of these markers in a biological sample may be evaluated by many methods. They may be evaluated for RNA expression levels. Hybridization methods are typically used, and may take the form of a PCR or related amplification method. Alternatively, a number of qualitative or quantitative hybridization methods may be used, typically with some standard of comparison, e.g., actin message. Alternatively, measurement of protein levels may performed by many means. Typically, antibody based methods are used, e.g., ELISA, radioimmunoassay, etc., which may not require isolation of the specific marker from other proteins. Other means for evaluation of expression levels may be applied upon purification of the marker. Antibody purification may be performed, though separation of protein from others, and evaluation of specific bands or peaks on protein separation may provide the same results. Thus, e.g., mass spectroscopy of a protein sample may indicate that quantitation of a particular peak will allow detection of the corresponding marker. Multidimensional protein separations may provide for quantitation of specific purified entities.

Tables 1A-C describe markers of the invention useful for the prognosis of prostate cancer.

Table 1A shows radical prostatectomy samples that were analyzed using the Eos Hu03 GENECHIP®, which contains 59680 probesets. Each probeset's intensity measure was entered as a continuous explanatory variable in a Cox proportional hazards regression survival analysis predicting relapse. Pretreatment PSA concentration was entered as a predictor in each analysis. The interquartile range hazard ratio (IQR HR) for each probeset was then calculated. This approach was used since in conventional Cox proportional hazards analyses, the hazards ratios for a covariate are computed by raising e, the base of natural logarithms, to the power of its regression coefficient. However, because the expression data are treated here as continuous covariates, hazards ratios expressed in this manner illustrate only the change in risk of relapse associated with a change of 1 unit on the expression scale, a change too small to be meaningful. To put the hazard ratios and associated confidence limits on a more interpretable scale, presented here is the hazards ratio associated with a change in expression values equivalent to 1 interquartile range (IQR) of the sample data for each probeset. The IQR is simply the 75th percentile minus the 25th percentile, and thus contains the middle 50 percent of observations. From this analysis, 266 probesets were found to be significant predictors of relapse at P<0.01.

Table 1B lists the accession numbers for Pkey's lacking UnigeneID's for table 1A. For each probeset is listed the gene cluster number from which oligonucleotides were designed. Gene clusters were compiled using sequences derived from Genbank ESTs and mRNAs. These sequences were clustered based on sequence similarity using Clustering and Alignment Tools (DoubleTwist, Oakland Calif.). Genbank accession numbers for sequences comprising each cluster are listed in the “Accession” column.

Table 1C shows genomic positioning for those Pkey's lacking Unigene ID's and accession numbers in table 1A. For each predicted exon, is listed the genomic sequence source used for prediction. Nucleotide locations of each predicted exon are also listed.

TABLE 1A

| Pkey | ExAccn | UnigeneID | Unigene Title | p value |
|---|---|---|---|---|
| 428664 | AK001666 | Hs.189095 | similar to SALL1 (sal (Drosophila)-like | 3.80177E−05 |
| 439785 | AA845608 | Hs.132860 | ESTs | 0.000106034 |
| 413924 | AL119964 | Hs.75616 | seladin-1 | 0.000157824 |
| 459680 | H96982 | Hs.42321 | ESTs | 0.00019382 |
| 431542 | H63010 | Hs.5740 | ESTs | 0.000250668 |
| 404824 | | | C22000161*: gi\|2443331\|dbj\|BAA22375.1\| (D [symbol] | 0.000290214 |
| 446021 | BE389213 | Hs.286 | ribosomal protein L4 | 0.000320882 |
| 434999 | AW975059 | | gb: EST387164 MAGE resequences, MAGN Homo | 0.000341555 |
| 458509 | AA654650 | Hs.282906 | ESTs | 0.000351184 |
| 406722 | H27498 | Hs.293441 | Homo sapiens SNC73 protein (SNC73) mRNA, | 0.000536315 |
| 423381 | BE250014 | | gb: 600943007F1 NIH_MGC_15 Homo sapiens c [symbol] | 0.000602528 |
| 419037 | R39895 | Hs.257391 | hypothetical protein DKFZp761J1523 | 0.00065526 |
| 414898 | AA157726 | Hs.264330 | N-acylsphingosine amidohydrolase (acid c [symbol] | 0.000707085 |
| 404582 | | | Target Exon | 0.00074185 |
| 458607 | AV656002 | | ESTs, Moderately similar to unnamed prot [symbol] | 0.000805762 |
| 402861 | D14661 | | Wilms' tumour 1-associating protein | 0.000870602 |
| 441494 | AW452344 | Hs.129977 | ESTs | 0.000875883 |
| 452753 | AA028049 | Hs.277728 | SEC14 (S. cerevisiae)-like 2 | 0.000934337 |
| 422516 | BE258862 | Hs.117950 | multifunctional polypeptide similar to S | 0.000969694 |
| 443675 | AI081397 | | ESTs | 0.000984435 |
| 425297 | AA354685 | | gb: EST63062 Jurkat T-cells V Homo sapien | 0.001036315 |
| 419517 | AF052107 | Hs.90797 | Homo sapiens clone 23620 mRNA sequence | 0.001065289 |
| 441345 | AW068579 | Hs.7780 | Homo sapiens mRNA; cDNA DKFZp564A072 (fr [symbol] | 0.00111943 |
| 438611 | AW204707 | Hs.123387 | ESTs | 0.001135255 |
| 434949 | AW976087 | | ESTs, Highly similar to AF161437 1 HSPC3 | 0.001142057 |
| 430845 | AF024690 | Hs.248056 | G protein-coupled receptor 43 | 0.001172874 |
| 429446 | AI547111 | | gb: PN2.1_A01_G12.r mynorm Homo sapiens c [symbol] | 0.001185816 |
| 444773 | BE156256 | Hs.11923 | hypothetical protein | 0.001200592 |
| 446702 | R44518 | Hs.143496 | ESTs | 0.001311934 |
| 415179 | D80630 | | gb: HUM091D02B Human fetal brain (TFujiwa [symbol] | 0.0013887 |
| 448479 | H96115 | Hs.21293 | UDP-N-acteylglucosamine pyrophosphorylas | 0.001402576 |
| 430799 | C19035 | Hs.164259 | ESTs | 0.001404901 |
| 454930 | AW845987 | Hs.68864 | ESTs, Weakly similar to phosphatidylseri | 0.001417466 |
| 407241 | M34516 | | gb: Human omega light chain protein 14.1 | 0.001504145 |
| 421970 | AF227156 | Hs.110103 | RNA polymerase I transcription factor RR | 0.001519398 |
| 434808 | AF155108 | Hs.256150 | Homo sapiens, Similar to RIKEN cDNA 2810 | 0.001610938 |
| 400207 | | | Eos Control | 0.00161581 |
| 423318 | AW467064 | Hs.5740 | ESTs | 0.001622161 |
| 413102 | AI199981 | Hs.109694 | ESTs, Weakly similar to T27691 hypotheti [symbol] | 0.001683835 |
| 411630 | U42349 | Hs.71119 | Putative prostate cancer tumor suppresso [symbol] | 0.001688301 |
| 419872 | AI422951 | Hs.146162 | ESTs | 0.001710345 |
| 402812 | | | NM_004930*: Homo sapiens capping protein | 0.001742994 |
| 427418 | AA402587 | | LAT1-3TM protein | 0.001743363 |
| 416276 | U41060 | Hs.79136 | LIV-1 protein, estrogen regulated | 0.001830512 |
| 457397 | AW969025 | Hs.109154 | ESTs | 0.001994494 |
| 403372 | AW249152 | | sirtuin (silent mating type information | 0.002012497 |
| 415344 | T65456 | | gb: yc73a11.r1 Soares infant brain 1NIB H | 0.002025172 |
| 422017 | NM003877 | Hs.110776 | STAT induced STAT inhibitor-2 | 0.002053043 |
| 406554 | | | Target Exon | 0.002105231 |
| 446057 | AI420227 | Hs.149358 | ESTs, Weakly similar to A46010 X-linked | 0.002151173 |
| 407040 | X03689 | | gb: Human mRNA fragment for elongation fa [symbol] | 0.002199926 |
| 419657 | AK001043 | Hs.92033 | integrin-linked kinase-associated serine | 0.002290654 |
| 457662 | AA907734 | Hs.124895 | ESTs | 0.002413693 |
| 447308 | AI005334 | Hs.22015 | ESTs, Weakly similar to 138344 titin, ca | 0.002472822 |
| 420707 | BE312807 | Hs.143407 | ESTs, Weakly similar to A54849 collagen | 0.002479439 |
| 426429 | X73114 | Hs.169849 | myosin-binding protein C, slow-type | 0.00251185 |
| 429289 | AI400746 | Hs.62187 | phosphatidylinositol glycan, class K | 0.002513019 |
| 454275 | AW293900 | Hs.304842 | ESTs, Weakly similar to AMYH_YEAST GLUCO [symbol] | 0.002559888 |
| 408603 | R25283 | Hs.326416 | Homo sapiens mRNA; cDNA DKFZp564H1916 (f [symbol] | 0.002571063 |
| 434614 | AI249502 | Hs.29669 | ESTs | 0.002629652 |
| 406558 | | | C5000893: gi\|6226859\|P38525\|EFG_THEMA | 0.002723963 |
| 440325 | NM003812 | Hs.7164 | a disintegrin and metalloproteinase doma | 0.002768837 |
| 440518 | AA888046 | Hs.233235 | ESTs | 0.002805131 |
| 424099 | AF071202 | Hs.139336 | ATP-binding cassette, sub-family C (CFTR | 0.002848507 |
| 421655 | AA464812 | | gb: zw63h05.r1 Soares_total_fetus_Nb2HF8_{—} | 0.002855486 |
| 445375 | AW779857 | Hs.166987 | ESTs, Weakly similar to B35363 synapsin | 0.002861874 |
| 456647 | AI252640 | Hs.110364 | peptidylprolyl isomerase C (cyclophilin | 0.002867794 |
| 433293 | AF007835 | Hs.32417 | hypothetical protein MGC4309 | 0.002897453 |
| 430389 | AL117429 | Hs.240845 | DKFZP434D146 protein | 0.002920262 |
| 423479 | NM014326 | Hs.129208 | death-associated protein kinase 2 | 0.00294831 |
| 443884 | N20617 | Hs.194397 | leptin receptor | 0.002997251 |
| 457926 | AA452378 | | Homo sapiens mRNA; cDNA DKFZp547J125 (fr [symbol] | 0.003054911 |
| 459710 | AI701596 | Hs.121592 | ESTs | 0.003061123 |
| 404560 | | | Target Exon | 0.003092402 |
| 438657 | AI141396 | Hs.158741 | ESTs | 0.003131957 |
| 400282 | | | NM_005313: Homo sapiens glucose regulated | 0.003134356 |
| 416144 | AA381556 | Hs.331803 | heat shock 60 kD protein 1 (chaperonin) | 0.003162736 |
| 430677 | Z26317 | | desmoglein 2 | 0.003170664 |
| 423562 | AJ005197 | Hs.7984 | pleckstrin homology, Sec7 and coiled/coi | 0.003217503 |
| 401040 | | | C11000425: gi\|4507721\|ref\|NP_003310.1\|ti | 0.003244184 |
| 419733 | AW362955 | | Homo sapiens cDNA FLJ14415 fis, clone HE | 0.003251143 |
| 415439 | R21114 | Hs.21383 | ESTs | 0.003317352 |
| 458054 | AW979052 | Hs.5734 | meningioma expressed antigen 5 (hyaluron [symbol] | 0.003355436 |
| 435346 | AI248389 | Hs.188105 | ESTs | 0.00337758 |
| 410452 | AW749026 | | gb: RC3-BT0319-100100-012-b05 BT0319 Homo | 0.003407284 |
| 427548 | AA813784 | Hs.123001 | ESTs | 0.003456322 |
| 438918 | AI126484 | Hs.127486 | ESTs | 0.00347913 |
| 448076 | AJ133123 | Hs.20196 | adenylate cyclase 9 | 0.003583335 |
| 420339 | AW968259 | Hs.186647 | ESTs | 0.003607275 |
| 426514 | BE616633 | Hs.170195 | bone morphogenetic protein 7 (osteogenic | 0.003628615 |
| 452143 | N29649 | Hs.260855 | Homo sapiens cDNA: FLJ21410 fis, clone C | 0.003701377 |
| 422813 | AV656571 | Hs.121068 | transmembrane 4 superfamily member 6 | 0.00379349 |
| 401524 | | | Target Exon | 0.003793904 |
| 453768 | BE382670 | Hs.198511 | Homo sapiens mRNA; cDNA DKFZp761I177 (fr [symbol] | 0.003810346 |
| 424954 | NM000546 | Hs.1846 | tumor protein p53 (Li-Fraumeni syndrome) | 0.003826169 |
| 440409 | AW294316 | Hs.125608 | ESTs | 0.003879241 |
| 452286 | AI358570 | Hs.123933 | ESTs, Weakly similar to ZN91_HUMAN ZINC | 0.003898535 |
| 444756 | AA278501 | Hs.200332 | hypothetical protein FLJ20651 | 0.003922529 |
| 429769 | NM004917 | Hs.218366 | kallikrein 4 (prostase, enamel matrix, p | 0.003947007 |
| 443403 | R01027 | Hs.133560 | ESTs | 0.003959306 |
| 400219 | | | Eos Control | 0.003966793 |
| 448489 | AI523875 | | gb: tg97d04.x1 NCI_CGAP_CLL1 Homo sapiens | 0.004120703 |
| 428378 | AA427571 | Hs.98531 | ESTs | 0.004121896 |
| 449909 | AA004681 | Hs.59432 | ESTs | 0.004158168 |
| 425127 | AW841272 | Hs.330418 | Homo sapiens cDNA: FLJ22459 fis, clone H | 0.004166839 |
| 427485 | AF039652 | Hs.178655 | ribonuclease H1 | 0.004198226 |
| 416305 | AU076628 | Hs.79187 | coxsackie virus and adenovirus receptor | 0.004214942 |
| 415075 | L27479 | Hs.77889 | Friedreich ataxia region gene X123 | 0.00422178 |
| 414091 | T83742 | Hs.334616 | gb: yd67g02.s1 Soares fetal liver spleen | 0.004236934 |
| 446415 | T27097 | Hs.22790 | ESTs | 0.004250994 |
| 407218 | AA095473 | Hs.28505 | ubiquitin-conjugating enzyme E2H (homolo [symbol] | 0.004267222 |
| 436626 | W35362 | Hs.103012 | ESTs | 0.00432651 |
| 448519 | AW175665 | Hs.278695 | Homo sapiens prostein mRNA, complete cds | 0.004332167 |
| 409841 | AW502139 | | gb: UI-HF-BR0p-ajr-e-05-0-UI.r1 NIH_MGC_5 | 0.004357117 |
| 423022 | AA320525 | Hs.201076 | ESTs | 0.004401104 |
| 429332 | AF030403 | Hs.199263 | Ste-20 related kinase | 0.004405129 |
| 417834 | BE172058 | Hs.82689 | tumor rejection antigen (gp96) 1 | 0.004424022 |
| 419808 | AW008030 | Hs.337536 | Homo sapiens cDNA: FLJ21568 fis, clone C | 0.004471786 |
| 450088 | AW292933 | Hs.254110 | ESTs | 0.004491465 |
| 431151 | BE207083 | | gb: ba10d10.y1 NIH-MGC_7 Homo sapiens cDN | 0.00450798 |
| 431281 | AW970573 | | gb: EST382654 MAGE resequences, MAGK Homo | 0.004657684 |
| 420960 | Z45662 | Hs.90797 | Homo sapiens clone 23620 mRNA sequence | 0.004798622 |
| 409540 | AW409569 | Hs.101550 | gb: fh01e09.x1 NIH_MGC_17 Homo sapiens cD | 0.004819322 |
| 456643 | AW751497 | Hs.98370 | cytochrome P450, subfamily IIS, polypept | 0.004821217 |
| 449889 | AA004613 | Hs.168672 | ESTs | 0.004888264 |
| 413074 | AI871368 | Hs.8417 | hypothetical protein DKFZp761M0423 | 0.004890295 |
| 452099 | BE612992 | Hs.27931 | hypothetical protein FLJ10607 similar to | 0.004925393 |
| 434263 | N34895 | Hs.44648 | ESTs | 0.004967084 |
| 400296 | AA305627 | Hs.139336 | ATP-binding cassette, sub-family C (CFTR | 0.004996569 |
| 435981 | H74319 | Hs.188620 | ESTs | 0.005005242 |
| 409430 | R21945 | Hs.346735 | splicing factor, arginine/serine-rich 5 | 0.005047202 |
| 414916 | AA206991 | | high-mobility group (nonhistone chromoso [symbol] | 0.005130846 |
| 434855 | AA765019 | Hs.191850 | ESTs | 0.005199586 |
| 406651 | AI559224 | | gb: tq32c02.x1 NCI_CCAP_Ut1 Homo sapiens | 0.005212356 |
| 440675 | AW005054 | Hs.47883 | ESTs, Weakly similar to KCC1_HUMAN CALCI [symbol] | 0.005249269 |
| 437412 | BE069288 | Hs.34744 | Homo sapiens mRNA; cDNA DKFZpS47C136 (fr [symbol] | 0.005270232 |
| 400487 | | | ENSP00000238977*: Interferon-induced prot [symbol] | 0.005353963 |
| 443366 | AI053501 | Hs.278869 | ESTs, Moderately similar to 2109260A B c | 0.005371997 |
| 410054 | AL120050 | Hs.58220 | Homo sapiens cDNA: FLJ23005 fis, clone L | 0.005404329 |
| 409344 | R47279 | Hs.285673 | hypothetical protein FLJ20950 | 0.005429984 |
| 421215 | AI868634 | Hs.246358 | ESTs, Weakly similar to T32250 hypotheti [symbol] | 0.005442884 |
| 450661 | AW952160 | | ESTs | 0.005447857 |
| 424269 | AW137691 | Hs.104696 | ESTs | 0.005483308 |
| 412294 | AA689219 | | poly(A)-binding protein, nuclear 1 | 0.005530138 |
| 404511 | | | NM_004349: Homo sapiens core-binding fact | 0.005558982 |
| 437006 | AW976322 | Hs.291561 | ESTs | 0.005639929 |
| 432989 | NM014074 | | PRO0529 protein | 0.00572161 |
| 417584 | AA252468 | Hs.1098 | DKFZp434J1813 protein | 0.005734515 |
| 437992 | AW450086 | Hs.145989 | ESTs, Highly similar to DHHC-domain-cont [symbol] | 0.005769051 |
| 447506 | R78778 | Hs.29808 | Homo sapiens cDNA: FLJ21122 fis, clone C | 0.005799441 |
| 420929 | AI694143 | Hs.296251 | programmed cell death 4 | 0.00585145 |
| 415121 | D60971 | Hs.34955 | Homo sapiens cDNA FLJ13485 fis, clone PL | 0.005963023 |
| 404662 | | | Target Exon | 0.006001874 |
| 445878 | AI262974 | Hs.145587 | ESTs | 0.006055258 |
| 421090 | BE301870 | Hs.101813 | solute carrier family 9 (sodium/hydrogen | 0.006079413 |
| 405155 | | | Target Exon | 0.006110052 |
| 427379 | D79254 | Hs.256066 | ESTs | 0.006133565 |
| 412561 | NM002286 | Hs.74011 | lymphocyte-activation gene 3 | 0.006142277 |
| 434257 | AF121255 | Hs.193053 | eukaryotic translation initiation factor | 0.006144213 |
| 400141 | | | Eos Control | 0.006200101 |
| 453359 | AA448787 | Hs.24872 | ESTs | 0.006315475 |
| 433151 | AW973735 | Hs.17631 | hypothetical protein DKFZp434E2135 | 0.006324267 |
| 449791 | AI248740 | Hs.133323 | ESTs | 0.006355539 |
| 405722 | BE410124 | | NM_021120: Homo sapiens discs, large (Dro [symbol] | 0.006388997 |
| 427527 | AI809057 | Hs.293441 | immunoglobulin heavy constant mu | 0.006397862 |
| 411487 | AF116666 | Hs.70333 | hypothetical protein MGC10753 | 0.006474544 |
| 417407 | AA923278 | Hs.290905 | ESTs, Weakly similar to protease [H.sapi [symbol] | 0.00651405 |
| 437233 | D81448 | Hs.339352 | Homo sapiens brother of CDO (BOC) mRNA, | 0.006535001 |
| 443425 | AI056776 | Hs.133397 | ESTs, Weakly similar to I78885 serine/th | 0.006574089 |
| 409179 | BE062633 | Hs.28338 | KIAA1546 protein | 0.006647277 |
| 431947 | AL359613 | Hs.49933 | hypothetical protein DKFZp762D1011 | 0.006663987 |
| 402339 | | | NM_003425*: Homo sapiens zinc finger prot [symbol] | 0.006744987 |
| 422262 | AL022315 | Hs.113987 | lectin, galactoside-binding, soluble, 2 | 0.006803463 |
| 404458 | | | CX000877*: gi\|11877268\|emb\|CAC18893.1\|(A [symbol] | 0.006816499 |
| 431693 | AI459519 | | serine (or cysteine) proteinase inhibito [symbol] | 0.006849491 |
| 428734 | BE303044 | Hs.192023 | eukaryotic translation initiation factor | 0.00696046 |
| 444204 | AI129194 | Hs.143040 | ESTs | 0.007032748 |
| 406837 | R70292 | Hs.156110 | immunoglobulin kappa constant | 0.007051544 |
| 442482 | NM014039 | Hs.8360 | PTD012 protein | 0.007051611 |
| 412006 | AW451618 | | ESTs | 0.00705506 |
| 435354 | AA678267 | Hs.117115 | ESTs | 0.007095576 |
| 403505 | M97639 | | receptor tyrosine kinase-like orphan rec | 0.007139282 |
| 451946 | AI824901 | Hs.281012 | ESTs, Highly similar to strong homology | 0.007271734 |
| 433339 | AF019226 | Hs.8036 | glioblastoma overexpressed | 0.007286776 |
| 436924 | AA741001 | Hs.326006 | ESTs | 0.007312314 |
| 431578 | AB037759 | Hs.261587 | GCN2 elF2alpha kinase | 0.007346563 |
| 419551 | AW582256 | Hs.91011 | anterior gradient 2 (Xenepus laevis) hom | 0.007352833 |
| 434256 | AI378817 | Hs.191847 | ESTs | 0.00736484 |
| 439778 | AL109729 | Hs.99364 | putative transmembrane protein | 0.0073683 |
| 423443 | AI432601 | Hs.168812 | Homo sapiens cDNA FLJ14132 fis, clone MA | 0.007425186 |
| 405293 | | | Target Exon | 0.007457507 |
| 426357 | AW753757 | Hs.12396 | gb: RC3-CT0283-271099-021-a08 CT0283 Homo | 0.007488395 |
| 422921 | BE062045 | | Homo sapiens cDNA: FLJ23260 fis, clone C | 0.007499187 |
| 417501 | AL041219 | Hs.82222 | sema domain, immunoglobulin domain (Ig), | 0.007512156 |
| 426091 | BE544541 | Hs.249495 | heterogeneous nuclear ribonucleoprotein | 0.007576069 |
| 416974 | AF010233 | Hs.80667 | RALBPI associated Eps domain containing | 0.007594318 |
| 449787 | AA005341 | Hs.283559 | ESTs | 0.007675199 |
| 412162 | AA100600 | Hs.69192 | gb: zn63b10.s1 Stratagene HeLa cell s3 93 | 0.007681586 |
| 413522 | BE145897 | | gb: MRO-HT0208-221299-204-b07 HT0208 Homo | 0.007824405 |
| 426788 | U66615 | Hs.172280 | SWI/SNF related, matrix associated, acti [symbol] | 0.007843962 |
| 414586 | AA306160 | Hs.76506 | lymphocyte cytosolic protein 1 (L-plasti [symbol] | 0.007931767 |
| 450382 | AA397658 | Hs.60257 | Homo sapiens cDNA FLJ13598 fis, clone PL | 0.007975007 |
| 404242 | | | ENSP00000252213*: SODIUM BICARBONATE COTR | 0.008032744 |
| 400206 | | | Eos Control | 0.008161865 |
| 441011 | AW137447 | Hs.126408 | ESTs | 0.008169197 |
| 449223 | AB002348 | Hs.23263 | KIAA0350 protein | 0.008169995 |
| 451776 | W45679 | Hs.169854 | hypothetical protein SP192 | 0.008174536 |
| 418354 | BE386973 | Hs.84229 | splicing factor, arginine/serine-rich 8 | 0.00821493 |
| 435188 | AA669512 | Hs.116679 | ESTs, Weakly similar to A42826T-cell le [symbol] | 0.00826337 |
| 415457 | AW081710 | Hs.7369 | ESTs, Weakly similar to ALU1_HUMAN ALU S | 0.008283276 |
| 432981 | NM002733 | Hs.3136 | protein kinase, AMP-activated, gamma 1 n | 0.008309431 |
| 433468 | AA832055 | Hs.170222 | ESTs, Weakly similar to ALU1_HUMAN ALU S | 0.008310151 |
| 457269 | AI338993 | Hs.134535 | ESTs | 0.00834154 |
| 431676 | AI685464 | | gb: tt88f04.x1 NCI_CGAP_Pr28 Homo sapiens | 0.008414644 |
| 426501 | AW043782 | Hs.293616 | ESTs | 0.008416828 |
| 447623 | AA350235 | Hs.6127 | Homo sapiens cDNA: FLJ23020 fis, clone L | 0.008419744 |
| 429678 | N70394 | Hs.238956 | ESTs | 0.008452349 |
| 444370 | NM015344 | Hs.11000 | leptin receptor overlapping transcript-1 | 0.00847352 |
| 404557 | | | C8001174*: gi\|10432400\|emb\|CAC10290.1\|(A [symbol] | 0.008502518 |
| 422867 | L32137 | Hs.1584 | cartilage oligomeric matrix protein (pse [symbol] | 0.008537039 |
| 441283 | AA927670 | Hs.131704 | ESTs | 0.008562466 |
| 424640 | AA344559 | Hs.164428 | ESTs | 0.008568818 |
| 452793 | AW138760 | Hs.61484 | ESTs | 0.008570907 |
| 420527 | AA332287 | Hs.175110 | ESTs | 0.00858412 |
| 421515 | YI1339 | Hs.105352 | GalNAc alpha-2, 6-sialyltransferase I, 1 | 0.008588847 |
| 430316 | NM000875 | Hs.239176 | insulin-like growth factor 1 receptor | 0.008606329 |
| 436524 | AA922236 | Hs.221037 | ESTs | 0.008616325 |
| 444700 | NM003645 | Hs.11729 | fatty-acid-Coenzyme A ligase, very long- | 0.008668985 |
| 441222 | AI277237 | Hs.44208 | hypothetical protein FLJ23153 | 0.008703638 |
| 429170 | NM001394 | Hs.2359 | dual specificity phosphatase 4 | 0.008704913 |
| 454393 | BE153288 | | gb: PM0-HT0335-180400-008-c08 HT0335 Homo | 0.008716471 |
| 456107 | AA160000 | Hs.137396 | ESTs, Weakly similar to JC5238 galactosy | 0.008767147 |
| 402091 | | | Target Exon | 0.008853214 |
| 409115 | AI223335 | Hs.50651 | Janus kinase 1 (a protein tyrosine kinas | 0.008866852 |
| 423250 | BE061916 | Hs.125849 | chromosome 8 open reading frame 2 | 0.008901601 |
| 428944 | AA780181 | Hs.41182 | Homo sapiens DC47 mRNA, complete cds | 0.008970935 |
| 419052 | T83291 | Hs.220624 | ESTs | 0.008998014 |
| 446203 | Z47553 | Hs.14286 | flavin containing monooxygenase 5 | 0.009023814 |
| 428180 | AI129767 | Hs.182874 | guanine nucleotide binding protein (G pr [symbol] | 0.009035339 |
| 452264 | AU077013 | Hs.28757 | transmembrane 9 superfamily member 2 | 0.009036494 |
| 446425 | AW295364 | Hs.255418 | ESTs | 0.009058296 |
| 446547 | AI334965 | Hs.176976 | ESTs | 0.009087495 |
| 419555 | AA244416 | | gb: nc07d11.s1 NCI_CGAP_Prl Homo sapiens | 0.009114049 |
| 422068 | AI807519 | Hs.104520 | Homo sapiens cDNA FLJ13694 fis, clone PL | 0.009119167 |
| 434826 | AF155661 | Hs.22265 | pyruvate dehydrogenase phosphatase | 0.009188183 |
| 411950 | T28407 | Hs.81564 | platelet factor 4 | 0.009188186 |
| 457146 | BE271371 | | biphenyl hydrolase-like (serine hydrolas [symbol] | 0.009228646 |
| 454131 | AI215902 | Hs.88845 | ESTs, Highly similar to T50835 hypotheti [symbol] | 0.009282618 |
| 404483 | | | C8000657*: gi\|1504040\|dbj\|BAA13219.1\|(D8 | 0.009290064 |
| 421351 | AU076667 | Hs.103755 | receptor-interacting serine-threonine ki | 0.00929738 |
| 417963 | AA210718 | Hs.104157 | ESTs, Weakly similar to KIAA0694 protein | 0.009334158 |
| 429011 | AA443182 | Hs.188835 | ESTs | 0.009370261 |
| 425380 | AA356389 | Hs.32148 | AD-015 protein | 0.009402223 |
| 442315 | AA173992 | Hs.7956 | ESTs, Moderately similar to ZN91_HUMAN Z | 0.009446269 |
| 424546 | BE465173 | Hs.194031 | ESTs | 0.009446339 |
| 444524 | AI160643 | Hs.28332 | Homo sapiens cDNA: FLJ21560 fis, clone C | 0.009472535 |
| 408446 | AW450669 | Hs.45068 | hypothetical protein DKFZp434I143 | 0.009508794 |
| 422669 | H12402 | Hs.119122 | ribosomal protein L13a | 0.00950994 |
| 420593 | AA280356 | Hs.187634 | ESTs | 0.009517511 |
| 447502 | AA312531 | Hs.26471 | Bardet-Biedl syndrome 4 | 0.0096083 |
| 412825 | AW167439 | Hs.190651 | Homo sapiens cDNA FLJ13625 fis, clone PL | 0.009645426 |
| 434401 | AI864131 | Hs.71119 | Putative prostate cancer tumor suppresso [symbol] | 0.009778291 |
| 432826 | X75363 | Hs.250770 | ACO for serine protease homologue | 0.009849589 |
| 428840 | M15990 | Hs.194148 | v-yes-1 Yamaguchi sarcoma viral oncogene | 0.009881804 |
| 413592 | AA130654 | Hs.302274 | Homo sapiens cDNA FLJ12328 fis, clone MA | 0.009899125 |
| 443102 | AI247472 | Hs.132965 | ESTs | 0.009964996 |

Pkey: Unique Eos probeset identifier number
ExAccn: Exemplar Accession number, Genbank accession number
UnigeneID: Unigene number
Unigene Title: Unigene gene title
p value: p value for relapse prediction (see Table 1A description)

TABLE 1B

| Pkey | CAT Number | Accession |
|---|---|---|
| 409841 | 1156088_1 | AW502139 AW502432 AW502235 AW501683 AW502647 |
| 410452 | 1204142_1 | AW749026 BE066111 T97135 |
| 412006 | 127108_1 | AW451618 AA846096 AI004201 AI242026 N38791 AI032976 AA099469 N45423 |
| 412294 | 128797_2 | AA689219 AI983045 T16928 Z45040 R20321 |
| 413522 | 1374614_1 | BE145897 BE145816 BE145885 |
| 414916 | 15071_24 | AA206991 BE564126 AA092392 AA090034 AA090545 AA093840 N84434 BE269369 |
| | | AI535705 AI535744 AI535682 AF283771 H28296 H27400 BE618821 AI873907 BE622711 |
| | | AI471738 AA557452 AA304303 AW794938 AA600212 AW027283 AW938645 AI654646 |
| | | AA370554 AA356536 AA715713 N87841 AW575412 AA987424 AA319424 BE084055 |
| | | AA827973 AA330422 AW630429 N38949 AA360952 AA045606 BE257213 AW768545 |
| | | AA101746 AI335554 N26696 AI630155 AW170282 AA206705 AA357094 AW603120 |
| | | AW793181 AI127978 AA639183 AW020136 BE536372 AA093946 AA730118 BE079411 |
| | | T90564 D83849 D20752 W07682 BE540914 F22618 AI041775 AA196344 AA366696 |
| | | AA083771 AA054783 AA330028 BE544267 AA247271 AW958331 BE073175 AW945457 |
| | | AA229491 AW874401 R34185 R81133 W32781 AI191194 BE277231 W79255 AW800102 |
| | | AI935842 AA928301 AA230310 AI742195 BE566990 AW673140 AI829489 AA054719 |
| | | AW512749 AA782987 AI088142 AW103898 AA714697 AW574795 AI056134 AW162373 |
| | | BE148890 AW068721 AW076120 AA563764 AW016252 AW016253 AI338171 AI085967 |
| | | AI338788 BE542084 AI186025 AI963188 AW079946 AI034040 AI961313 AI831345 N79755 |
| | | AA029435 AA910600 AA618386 AI336429 AA230308 AI346567 AA541647 AW024986 |
| | | AI926174 AA878167 AW026237 AA668251 W15170 AA129635 AI363729 AA309687 |
| | | AI453176 AI282417 H89557 AW264978 D55190 AA188911 AI471512 AI537126 AW675575 |
| | | AI673287 AI476121 AA563901 AA353344 N93269 N80559 L13805 AA564621 AI056119 |
| | | AI587020 AW874624 AI803890 AW074286 AA745955 AW152331 AI282228 AI081139 |
| | | AI147252 AI126996 AI970694 D55874 AA313759 AW023735 AA999920 AI285652 |
| | | AI476553 AI252804 AI096960 AW151090 AA876366 W32423 D57151 AA856637 AI954376 |
| | | W73923 AL047978 BE041344 AA861867 AI346526 AL047979 AI348036 AI187244 |
| | | AA328683 AA197248 N72984 AA862752 AA747207 AA876587 AA845496 AA890470 |
| | | AW170401 AI127224 N99881 AW074379 AA938114 AI197777 AI753834 AI346536 |
| | | AA331597 AI367738 AA977063 W93785 AA872167 AI932924 AA614560 AI434283 |
| | | AI160153 AW130136 BE542026 AA385117 AA130703 AA778269 AI769329 AI285034 |
| | | AW340835 AI224601 AA663430 AA846183 AI362627 AA903448 AW238760 AI283178 |
| | | AV662138 AI138363 AA860743 AI368179 AI280190 AI139131 AI359157 H99812 |
| | | AA771749 AI539068 AI089843 AI566789 AI281240 AI352354 AI769243 AI092187 |
| | | AI073627 AI473623 AW276039 AI798397 AI024587 AA889467 AI683918 AW673268 |
| | | AA602941 AA861823 AA668586 AA772542 AI077928 AA594116 AI018648 AI421799 |
| | | AA705955 AA586855 AA577106 AI131297 AI355412 AI350882 AW265014 AW043934 |
| | | AI127696 AW469864 AI041801 AL048264 AA961777 AI246050 AA566002 AI469308 |
| | | AA809086 AW768947 AA507781 AI361342 AI368477 AA133897 AI300444 AI768467 |
| | | AA773978 AW062352 AA648130 AA827606 AI094950 T61248 AA101747 AI348251 |
| | | AI092294 AA565522 T39158 W33201 C75489 AA670425 AA483085 R48684 T28966 |
| | | H96803 AA641999 AA709360 H99805 T19371 AW879059 AA524370 AW338262 N72895 |
| | | AW591714 T63777 AL047945 AA150131 AA146973 AW878989 AA877803 T56122 |
| | | AA147065 AA342484 AA342236 AW270920 AI913364 AW795486 AI865002 W94286 |
| | | AA209325 T40443 AI268918 AI418552 T48135 M62207 AA328164 AW795480 BE169953 |
| | | BE169983 AA206888 AA132394 AW149866 T57929 W15510 C75674 R81132 AI423687 |
| | | AI193465 H28297 AA994473 F04357 BE243460 AA987347 AI376779 AA927274 T03381 |
| | | H99134 T03851 AA384714 AW265058 BE041328 BE541757 AI910675 T64485 N89843 |
| | | AA688338 T64628 AI143530 AI026855 F03043 AA865434 AA363018 AA459233 AA664746 |
| | | N68567 AW467363 T16030 AW149914 AA994312 BE350136 AA307427 AI658528 L13804 |
| | | AA384004 N71219 N22172 AW364964 |
| 415179 | 1527481_1 | D80630 D80896 D80895 |
| 415344 | 1534510_1 | T65456 F11749 Z43023 F06216 R18181 R17246 |
| 419555 | 185884_1 | AA244416 AA244401 |
| 419733 | 187589_1 | AW362955 H59488 AI040666 W60959 W94209 H27231 T84625 H75715 W04957 W63676 |
| | | AA659693 AA514302 W63789 BE046412 T91396 AI951970 AW044233 N20018 AW663548 |
| | | T90114 AI139947 AA809643 AA846232 AA581966 AA789002 AA295134 AW188870 |
| | | H75644 AA526037 AA347970 AW961788 H61476 AL133779 AA449282 H28581 AA249370 |
| 421655 | 204993_1 | AA464812 AA431899 AA295193 AW959241 |
| 422921 | 222939_1 | BE062045 Z43804 W35143 AI761615 N33753 BE062044 BE551229 AI088004 N33865 |
| | | AA332473 AA374196 N48481 |
| 423381 | 227731_1 | BE250014 BE293608 BE252781 AA325222 AW904396 |
| 425297 | 249704_1 | AA354685 AW962101 H85269 F11427 R55281 |
| 427418 | 278594_1 | AA402587 AI760178 AI911270 AI184927 AI277654 AA402398 AI633280 AW002589 |
| | | AI984968 AI810234 AI671725 AI419580 AA705629 AW138044 AI719961 D45607 |
| | | AA455831 |
| 429446 | 304683_1 | AI547111 AW973749 AA558007 |
| 430677 | 3216_1 | Z26317 NM_001943 AW991316 BE018413 AW996800 AW996267 AW996264 W73983 |
| | | AA313797 BE513193 AW861416 AW857494 AA488331 BE171045 AW366926 BE002219 |
| | | AW996792 AW753487 AW361908 BE003946 AW858751 AW858747 AW858750 |
| | | AW858755 AW858749 D58979 AW363740 AW859003 AW363742 AW858999 AW471344 |
| | | BE072891 AW753745 BE395396 AI378517 D58730 AW748942 BE395765 BE153312 |
| | | BE153169 BE153241 AW371849 AW371853 AW748956 AA506621 AA723159 AI933746 |
| | | AW473996 AW572140 |
| 431151 | 328652_−1 | BE207083 |
| 431281 | 330904_1 | AW970573 AA501880 AA501870 |
| 431676 | 336411_1 | AI685464 AW971336 AA513587 AA525142 |
| 431693 | 33663_3 | AI459519 AI366092 AF121175 AL042956 F11899 AI436382 AI133591 AI675879 AA081306 |
| | | AI948730 BE243645 AA448957 H09862 AI382265 N92723 AL048959 AI356415 BE245782 |
| | | AI288626 AI949306 AI814412 AW207026 AI659678 AI984766 AA741391 AI453490 |
| | | AW166423 AI799883 AL045697 AI826075 AI952039 AA167742 BE463776 R01203 |
| | | AI972947 AI623819 AW167132 AW337996 AW264027 AA209462 AI863491 T65400 |
| | | AI394192 R62397 AW968250 BE464852 AW474624 AI758979 AW474705 BE046016 |
| | | AI949348 AI289432 AI620722 AW440580 AI610824 AI458169 AW002172 AI634183 |
| | | AA648408 AI289435 C00469 R62398 AI287482 H24845 F09546 AI125609 W93405 |
| | | AA150039 AA150203 H09775 AI951377 AI631154 AA258738 AA744971 AA449685 |
| | | AI434048 AA167836 R01316 T54772 |
| 432989 | 35719_1 | NM_014074 AF111848 |
| 434949 | 39603_1 | AW976087 AA100561 AF161437 D30850 AA767385 AI990080 AI337209 AA086348 |
| | | AW002909 AA747908 AW450816 AW361653 BE145974 BE146300 AW292658 |
| 434999 | 397353_1 | AW975059 AA659177 AA733194 |
| 443675 | 577019_1 | AI081397 N94610 AI633993 AW949183 W23817 AW297357 H17610 F32559 |
| 448489 | 765247_1 | AI523875 R45782 R45781 |
| 450661 | 84193_1 | AW952160 AI819147 AA774089 AA010589 AA319638 AI954753 AI634083 H39119 |
| | | AA812766 |
| 454393 | 115888_1 | BE153288 BE153151 BE152925 AA078302 |
| 457146 | 29193_1 | BE271371 NM_004332 X81372 AI167945 AW071802 AI818871 AI017491 AA421820 |
| | | AA558952 AA910750 AA973795 R54850 AI672895 AI418120 AI268326 AA911487 |
| | | AA167197 N46097 X57653 R10551 T28159 AA167111 AW840204 AW276222 R09405 |
| | | N46098 AA284554 AW129121 |
| 457926 | 43767_1 | AA452378 AL390181 H05571 R53363 R55079 R11987 R11919 R84811 R19546 AA046904 |
| | | R22842 AL134431 F11225 W79925 H10691 AA354088 AW965695 AI198775 AI803682 |
| | | AA040404 AI150653 AA040266 AI436656 AW575893 AI703024 AA446858 AI805847 |
| | | AI699312 AW575924 R55051 R53965 R39826 AW772031 AA975258 AW901905 R43388 |
| | | BE218163 AI074604 AI148281 AA758256 BE501159 H11032 AW131553 F08888 |
| | | AW341569 AI347996 AI952708 AI374835 AI089094 AI284927 W74206 AI027303 AI274177 |
| | | AW299757 AI377712 AW300882 AA883979 AI239912 AI346165 AA947211 R46050 |
| | | AI698833 AA452150 R43898 AA904733 |
| 458607 | 65602_1 | AV656002 AV655810 |

Pkey: Unique Eos probeset identifier number
CAT number: Gene cluster number
Accession: Genbank accession numbers

TABLE 1C

| Pkey | Ref | Strand | Nt_position |
|---|---|---|---|
| 400487 | 8919452 | Plus | 19369-20782 |
| 401040 | 7232177 | Plus | 17623-17919 |
| 401524 | 7770429 | Plus | 34644-35263 |
| 402091 | 8117554 | Minus | 190-306 |
| 402339 | 7459859 | Minus | 24698-26511 |
| 402812 | 6010110 | Plus | 25026-25091, 25844-25920 |
| 402861 | 2814366 | Minus | 14933-15231, 15387-15627 |
| 403372 | 9087278 | Minus | 130002-130131 |
| 403505 | 7577651 | Plus | 11059-11541 |
| 404242 | 5672600 | Minus | 22722-22897, 23164-23433 |
| 404458 | 7770571 | Minus | 35710-36276 |
| 404483 | 8096904 | Minus | 162212-163710 |
| 404511 | 8151864 | Minus | 148501-148659 |
| 404557 | 7243881 | Minus | 88508-88699 |
| 404560 | 8954219 | Plus | 29247-29437 |
| 404582 | 9739220 | Plus | 53230-53424 |
| 404662 | 9797105 | Minus | 99466-99713 |
| 404824 | 6478944 | Plus | 209436-209545, 209741-209850 |
| 405155 | 9966228 | Plus | 130469-130723 |
| 405293 | 3845419 | Minus | 16255-16535, 16665-17340 |
| 405722 | 9800078 | Plus | 140732-140892, 141099-141268, |
| | | | 141434-141714, 142048-142192 |
| 406554 | 7711566 | Plus | 106956-107121 |
| 406558 | 7711569 | Minus | 14052-14190 |

Pkey: Unique number corresponding to an Eos probeset
Ref: Sequence source. The 7 digit numbers in this colunm are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.
Note:
the ExAccn number of NM_is abbreviated to NM in Table 1A-C.

Table 2 lists the first 50 genes, ranked by P value, identified by survival analysis to be associated with prostate cancer relapse.

TABLE 2

| | UniGene | Genbank | | | |
|---|---|---|---|---|---|
| Rank | cluster | accession | Gene title | Risk of relapse^{a} | P |
| 1 | Hs.189095 | NM_020436 | Sal-like 4 | 2.040 | 0 |
| 2 | Hs.132860 | AA845608 | ESTs | 0.341 | 0 |
| 3 | Hs.75616 | NM_014762 | 24-Dehydrocholesterol reductase (seladin-1) | 0.293 | 0 |
| 4 | Hs.42321 | NM_173605 | Hypothetical protein LOC283518 | 2.133 | 0 |
| 5 | Hs.80667 | NM_004726 | RALBP1 associated Eps domain containing 2 (REPS2) | 0.172 | 0 |
| 6 | Hs.163543 | NM_144704 | Hypothetical protein FLJ30473 | 3.241 | 0 |
| 7 | Hs.286 | NM_000968 | Ribosomal protein L4 | 0.215 | 0 |
| 8 | Hs.114670 | D49387 | Leukotriene B4 12-hydroxydehydrogenase | 2.380 | 0 |
| 9 | Hs.366053 | NM_024080 | Transient receptor potential cation channel, subfamily M, member 8 (trp-p8) | 0.260 | 0 |
| 10 | Hs.366 | AL389978 | Immunoglobulin heavy chain variable region | 2.436 | 0.001 |
| 11 | Not available | BE250014 | ESTs | 0.295 | 0.001 |
| 12 | Hs.257391 | NM_032293 | Hypothetical protein DKFZp761J1523 | 3.138 | 0.001 |
| 13 | Hs.264330 | AK024677 | N-acylsphingosine amidohydrolase (acid ceramidase)-like | 0.256 | 0.001 |
| 14 | Hs.123468 | NM_033225 | CUB and Sushi multiple domains 1 | 0.185 | 0.001 |
| 15 | Not available | AV656002 | EST | 0.251 | 0.001 |
| 16 | Hs.129977 | AW452344 | ESTs | 0.229 | 0.001 |
| 17 | Hs.277728 | NM_012429 | SEC14-like 2 | 0.348 | 0.001 |
| 18 | Hs.117950 | NM_006452 | Phosphoribosylaminoimidazole carboxylase | 0.321 | 0.001 |
| 19 | Hs.424973 | BC018081 | Clone IMAGE: 4793702 | 0.225 | 0.001 |
| 20 | Not available | AA354685 | EST | 0.363 | 0.001 |
| 21 | Hs.356547 | NM_138799 | Hypothetical protein BC016005 | 0.337 | 0.001 |
| 22 | Hs.7780 | AL049969 | cDNA DKFZp564A072 | 0.186 | 0.001 |
| 23 | Hs.123387 | AW204707 | ESTs | 0.375 | 0.001 |
| 24 | Hs.377879 | AK055649 | cDNA FLJ31087 fis | 3.112 | 0.001 |
| 25 | Hs.248056 | NM_005306 | G protein-coupled receptor 43 | 0.211 | 0.001 |
| 26 | Hs.301947 | NM_014509 | Kraken-like serine hydrolase | 0.212 | 0.001 |
| 27 | Hs.11923 | NM_018982 | Hypothetical protein DJ167A19.1 | 0.155 | 0.001 |
| 28 | Hs.247423 | NM_001617 | Adducin 2 (β) (ADD2) | 2.044 | 0.001 |
| 29 | Not available | D80630 | EST | 2.753 | 0.001 |
| 30 | Hs.21293 | NM_003115 | UDP-N-acteylglucosamine pyrophosphorylase 1 | 0.185 | 0.001 |
| 31 | Hs.292859 | C19035 | ESTs, moderately similar to VPP2_HUMAN | 2.375 | 0.001 |
| 32 | Hs.68864 | AW845987 | Lipase, member H (LIPH), mRNA | 0.273 | 0.001 |
| 33 | Hs.405944 | X57819 | Ig λ chain | 2.388 | 0.002 |
| 34 | Hs.110103 | NM_018427 | RNA polymerase I transcription factor RRN3 | 0.337 | 0.002 |
| 35 | Hs.256150 | NM_080654 | NY-REN-41 antigen | 2.718 | 0.002 |
| 36 | Hs.76847 | NM_014610 | α Glucosidase II alpha subunit | 0.135 | 0.002 |
| 37 | Hs.109694 | AI199981 | Oxysterol binding protein-like 8 (OSBPL8), mRNA. | 4.511 | 0.002 |
| 38 | Hs.71119 | NM_006765 | Putative prostate cancer tumor suppressor (N33) | 0.281 | 0.002 |
| 39 | Hs.146162 | AK075364 | ESTs. | 2.151 | 0.002 |
| 40 | Hs.333417 | NM_004930 | Cappling protein (actin filament) muscle Z-line, β | 0.291 | 0.002 |
| 41 | Hs.410998 | AA402587 | ESTs, Highly similar to MLL septin-like fusion | 1.507 | 0.002 |
| 42 | Hs.79136 | NM_012319 | LIV-1 protein, estrogen regulated | 0.210 | 0.002 |
| 43 | Hs.109154 | AW969025 | ESTs | 0.281 | 0.002 |
| 44 | Hs.433622 | NM_007085 | Follistatin-like 1 (FSTL1) | 0.233 | 0.002 |
| 45 | Not available | T65456 | EST | 0.195 | 0.002 |
| 46 | Hs.405946 | NM_003877 | Suppressor of cytokine signaling 2 (SOCS2) | 0.448 | 0.002 |
| 47 | Hs.127699 | NM_001369 | Dynein, axonemal, heavy polypeptide 5 (DNAH5) | 0.284 | 0.002 |
| 48 | Hs.422118 | NM_001402 | Eukaryotic translation elongation factor 1 alpha 1 | 0.175 | 0.002 |
| 49 | Hs.92033 | NM_030768 | Integrin-linked kinase-associated serine/threonine phosphatase 2C | 5.564 | 0.002 |
| 50 | Hs.124895 | AA907734 | ESTs | 3.399 | 0.002 |

^{a}The risk of relapse is the IQR HR calculated for each probeset as described in “Materials and Methods.”

Sequences described therein, where incomplete, may be extended either by informatics techniques, or by techniques of biochemistry and molecular biology. Many well known methods are available. See, e.g., Mount (2001) Bioinformatics: Sequence and Genome Analysis CSH Press, NY; Baxevanis and Oeullette (eds. 1998) Bioinformatics: A Practical Guide to the Analysis of Genes and Proteins (2d. ed.) Wiley-Liss; Ausubel, et al. (eds. 1999 and supplements) Current Protocols in Molecular Biology Lippincott; and Sambrook, et al. (2001) Molecular Cloning: A Laboratory Manual (3d ed., Vol. 1-3) CSH Press.

Nucleic acid sequences are particularly described. Using linkages to publicly accessible databases, e.g., GenBank accession numbers, sequences are described whose presence or absence in the samples provides prognostic capacity. Correlations are made between the detection of such sequence and the outcomes of the prostate cancers. Thus, detection of physically linked, e.g., adjacent or contiguous, sequence will be equivalent. The correlation between presence of a 5′ segment will be equivalent to such with a 3′ segment of the same physical molecule.

The tables also provide protein sequences which correspond to the identified nucleic acid sequences. The amino acid embodiments of the markers will also exhibit similar correlations with outcome. Thus, the use of the protein embodiments can also be used in the invention. Proteins or fragments can be produced, and antibodies generated. See, e.g., Coligan (1991) Current Protocols in Immunology Lippincott; Harlow and Lane (1988) Antibodies: A Laboratory Manual CSH Press; and Goding (1986) Monoclonal Antibodies: Principles and Practice (2d ed.) Academic Press.

Kits for use in the prognostic methods are also made available. The kits will include reagents for detecting the markers, e.g., at the nucleic acid or protein level. Thus, for nucleic acid expression level prognosis kits, typically PCR primers or detectable hybridization probes will be included. For protein level prognosis kits, typically antibodies will be used to quantitate or detect the appropriate gene products. Typically instructions will be provided, which may include buffers or instructions for proper disposal of the materials.

Diagnostic, Therapeutic Applications

After prostate cancer has been identified, tests are performed to find out if cancer cells have spread within the prostate or to other parts of the body. Prostate cancer is typically classified into stages I-IV. The following tests and procedures may be used in the staging process: radionuclide bone scan, pelvic lymphadenectomy, CT scan, and seminal vesicle biopsy.

The list of targets may have other diagnostic applications besides outcome prediction. These identified markers may be valuable in such stage subsetting, distinct from outcome subsetting. Typically, after initial diagnosis, tests are performed to determine if cancer cells have spread within the prostate or to other parts of the body. Evaluation of the identified markers, singly or in combinations, may substitute for other tests to assign stage, or add to them for confirmation. Alternatively, the detection of one or more of these markers may be used as early detection screens for prostate cancer. Preferably, if the marker is soluble or released into a readily accessible body fluid, e.g., serum, semen, or urine, a diagnostic test for detection may allow for early detection of prostate cancer.

The invention is illustrated further by the following examples that are not to be construed as limiting the invention in scope to the specific procedures described in it.

### EXAMPLES

### Example 1

### Study Design

Tissue Collection and Preparation of RNA

A cohort of 72 fresh-frozen prostate cancers was collected from patients with localized prostate cancer treated by radical prostatectomy RP at St. Vincent's Hospital, Sydney. The primary outcome, disease-specific relapse, was measured from the date of RP and was defined as a rise in serum PSA above 0.3 ng/ml with subsequent further rises. Following inking of the external limits of the prostate immediately after removal and prior to formalin-fixation, up to six, 5 mm core biopsies were taken and stored at −80° C. for a later RNA extraction. The proportion of invasive cancer in the biopsy sample was then estimated retrospectively by either frozen sectioning of the biopsy and hematoxylin and eosin staining, or by examination of archival formalin-fixed, paraffin-embedded tissue surrounding the biopsy site. Only those biopsies that contained ≧75% invasive cancer were used for subsequent transcript profiling. Only one biopsy per patient was analyzed.

Xenograft Model

The androgen-dependent LuCaP-35 (7) prostate cancer xenograft was grown as subcutaneous tumors in nude male mice. To study the androgen-withdrawal process, tumor-bearing mice were castrated and monitored for tumor regression and PSA levels. Tumors were harvested from mice prior to castration, and at various time points (1-100 days) post-castration and were processed for microarray analysis. For data analysis and identification of androgen-regulated genes, the samples were binned in two groups consisting of days 0-2 and days 5-100 post-castration. Genes that showed a significant (P<0.01) difference in the means of each group were identified by a standard Student's t-Test.

RNA Extraction and Microarray Protocols

Preparation of total RNA from fresh-frozen prostate and xenograft tissue was performed by extraction with Trizol reagent (Life Technologies Inc., Gaithersburg, Md.) and was reverse transcribed using a primer containing oligo(dT) and a T7 promoter sequence. The resulting cDNAs were then in vitro transcribed in the presence of biotinylated nucleotides (Bio-11-CTP and Bio-16-UTP) using the T7 MEGAscript kit (Ambion, Austin, Tex.).

The biotinylated targets were hybridized to the Eos Hu03, a customized Affymetrix GENECHIP® (Affymetrix, Santa Clara, Calif.) oligonucleotide array comprising 59,619 probesets representing 46,000 unique sequences including both known and FGENESH predicted exons that were based on the first draft of the human genome. Hybridization signals were visualised using phycoerythrin-conjugated streptavidin (Molecular Probes, Eugene, Oreg.). Normalization of the data was performed as follows. The probe-level intensity data from each array were fitted to a fixed gamma distribution with a mean of 300 and a shape parameter of 0.81. This normalization procedure removes between chip variation attributable to non-biological factors. Then for each probeset, a single measure of average intensity was calculated using Tukey's trimean of the intensity of the constituent probes (8). Finally, a correction for nonspecific hybridization was applied, in which the average intensity measure of a “null” probeset consisting of probes with scrambled sequence was subtracted from all other probesets on the chip.

Statistical Methods

Prior to survival analysis, a screen was applied to the expression data to eliminate probesets without meaningful variation. For each probeset, the ratio of the 90^{th} percentile to the 15^{th} percentile intensity measure was required to be at least 2, and the minimum expression level was required to be at least 150 average intensity units. Separate Cox proportional hazards analyses with pretreatment PSA concentration dichotomised at 20 ng/ml and gene expression modeled as a continuous variable were used to identify gene expression that correlated with PSA recurrence (9). The IQR hazards ratio was computed by multiplying the regression coefficient for each probeset by its own interquartile range prior to exponentiation. The positive false discovery rate (pFDR) was calculated using the method described by Storey and Tibshirani (10). Schoenfeld residuals were used to assess the proportional hazards assumption for the two probesets for trp-p8 and the assumption was found to be upheld in both cases.

Variables of clinical relevance were also modeled in univariate analyses for their ability to predict disease-free survival in the 72 prostate cancers using the Cox proportional hazards model. Trp-p8 mRNA expression assessed by ISH, was reported as proportions within histological groups and compared between groups using a Fisher's Exact test. The expression dataset of 277 selected probesets from 72 samples was reordered according to cluster analysis in both dimensions (probesets and samples). In each analysis, the distance metric was the square root of (1−r), where r is the standard pearson product-moment correlation. The clustering algorithm used was Ward's minimum variance method (11).

In order to evaluate the ability of the 11 genes used by Singh et al., to accurately predict relapse status in aggregate in our dataset, we entered these eleven probesets into a multivariate Cox regression model, and used variable selection methods to choose a subset of predictors. Three different methods were used (forward selection, backward elimination, and stepwise selection), all using P=0.15 as inclusion/exclusion criterion). In each case, the final model using 4 probesets had a significance level of P=0.0029 by the likelihood ratio test.

All statistical analyses were performed using SAS (SAS Institute Inc., Cary, N.C.).

Tissue Microarray and In Situ Hybridization

Tissue microarrays were constructed as described previously (12), and were comprised of prostate cancer samples from 95 patients that are part of a previously published cohort of patients treated for localized prostate cancer by RP alone at St. Vincent's Hospital, Sydney (13). In addition, 13 prostate cancer specimens were collected from patients treated for localized prostate cancer by RP who had received at least 3 months (range 3-10 months) of preoperative neoadjuvant hormonal treatment (5 with anti-androgens alone, 6 with a combination of a Gn-RH analogue and anti-androgens and 2 with a Gn-RH analogue alone). Trp-p8 expression in these 13 samples was assessed on conventional tissue sections.

For ISH, a 424-base pair probe for trp-p8 was derived from the 3′ end of the trp-p8 gene and transcribed to produce a DIG-labeled riboprobe using an RNA DIG-labeling kit (Roche, y™ Mannheim, Germany). ISH was performed on the VENTANA DISCOVERY™ instrument (Ventana Medical Systems, Tucson, Ariz.) using the RIBOMAP™ kit with protease P2 for 2 minutes (Ventana Medical Systems, Tucson, Ariz.) and hybridization for 8 hours at 65° C. Chromogenic detection was achieved with the BLUEMAP™ detection system as described by the manufacturer (Ventana Medical Systems, Tucson, Ariz.).

### Example 2

### Expression Profiling of Prostate Cancers

In this study, we sought to discover novel biomarkers that might predict for PSA relapse following radical prostatectomy utilizing outcome-based statistical tools to analyze gene expression profiles of 72 prostate cancers. A criteria for selection was the ability to predict recurrence better than preoperative serum PSA concentration alone, since PSA is one of only a handful of markers that provide preoperative prognostic information. The 72 prostate tissues were collected at the time of radical prostatectomy (RP) from patients undergoing treatment for localized prostate cancer at St. Vincent's Hospital Campus, Sydney, Australia. At last follow-up (median=28.25 months, range 4.9-90.3 months), 17 of the 72 (23.6%) patients had relapsed, of which 14 demonstrated a rise in postoperative PSA levels while 3 patients were diagnosed with a rising PSA and local recurrence of disease. Consistent with published data (5, 6, 13), the significant predictors of prostate cancer relapse in this cohort on univariate analysis were Gleason score (HR=1.88, P=0.027), surgical margins (HR=4.90, P=0.035) and preoperative PSA concentration (HR=4.43, P=0.006) (Table 1). The overall relapse rate of 23.6% and median time to relapse of 14 months in this group of 72 patients was similar to that observed in a cohort of 732 patients treated for localized prostate cancer by RP at the same institution between 1986 and 1999 (13).

TABLE 3
Clinicopathological characteristics of the prostate cancer
cohort (n = 72) that were utilized in the survival analysis.

| Variable | HR (confidence levels) | P |
|---|---|---|
| Gleason score^{a} | 1.88 (1.08-3.29) | 0.027 |
| Preoperative PSA concentration | 4.43 (1.53-12.79) | 0.006 |
| <20 ng/ml vs. ≧20 ng/ml | | |
| Seminal vesicle involvement | 2.33 (0.88-6.14) | 0.086 |
| positive vs. negative | | |
| Surgical margins | 4.90 (1.12-21.5) | 0.035 |
| positive vs. negative | | |

^{a}Gleason score was modeled as a continuous variable.

RNA was extracted from a core biopsy taken at the time of RP for each of the 72 cases that comprised ≧75% cancer tissue. Biotinylated RNA from each sample was then analyzed with a customized GENECHIP® expression array, the Eos Hu03 (14). This single GENECHIP® microarray design is representative of greater than 90% of the expressed human genome based on the first public draft and comprises 59,619 probesets representative of both known and predicted genes (15). An initial screen was applied to the microarray probesets to choose genes expressed with reliable intensity and adequate cross-sample variance. This screen reduced the initial set of 59,619 probesets to a subset of 8,521 probesets for further examination.

### Example 3

### Survival Analysis

Each probeset's intensity value was entered as a continuous explanatory variable in a Cox proportional hazards survival analysis predicting relapse. Pretreatment PSA concentration was also entered as a predictor in each analysis. From this analysis, 264 probesets were found to be significant predictors of relapse at P<0.01. To assist interpretation, we next calculated the interquartile range hazard ratio (IQR HR) for each probeset. Because the expression data are treated here as continuous covariates, hazards ratios expressed in their natural scale illustrate only the change in risk of relapse associated with a change of 1 unit on the expression scale, a change too small to be comprehended easily. To put the hazard ratios and associated confidence limits on a more interpretable scale, we present here the hazards ratio associated with a change in expression values equivalent to 1 interquartile range (IQR) of the sample data for each probeset. The IQR is simply the 75th percentile minus the 25th percentile, and thus contains the middle 50 percent of observations.

The multiple hypothesis testing problem has been recognized as an important issue to address in microarray research. The large number of tests that are performed simultaneously on thousands of probesets greatly increases the chances of making Type I errors (or false-positive findings). To assess the effect of multiple hypothesis testing, we adapted a method developed by Storey and Tibshirani (2001) for calculating the positive false discovery rate (pFDR), an estimate of the proportion of false-positives present in a set of findings (10). This technique was developed explicitly for use with microarray data, for which the usual assumption of independence among tests is untenable. The procedure can be briefly summarized as follows. First, null data were simulated by randomly permuting the relapse status of subjects and re-performing the survival analyses. In each simulation, the number of relapsers and non-relapsers (17 and 55, respectively) remained constant, but these designations were shuffled and assigned to patients at random. The permutation was performed 500 times, and for each simulation, the number of findings at P<0.01 was noted. The mean number of findings across the 500 permutations was 85.9. This figure, an estimate of the expected number of false positives under null conditions, was then divided by the number of actual findings (n=264) to obtain an estimate of the proportion of false-positive findings. After the application of a correction factor (10), the final estimate for the pFDR was 23%. Thus, we can expect that approximately 61 of the 277 findings are false positives.

Identification of the Candidate Marker Genes

The 277 probesets (Table 1A-1C) identified by survival analysis included both known genes and hypothetical genes of unknown function, as well as ESTs.

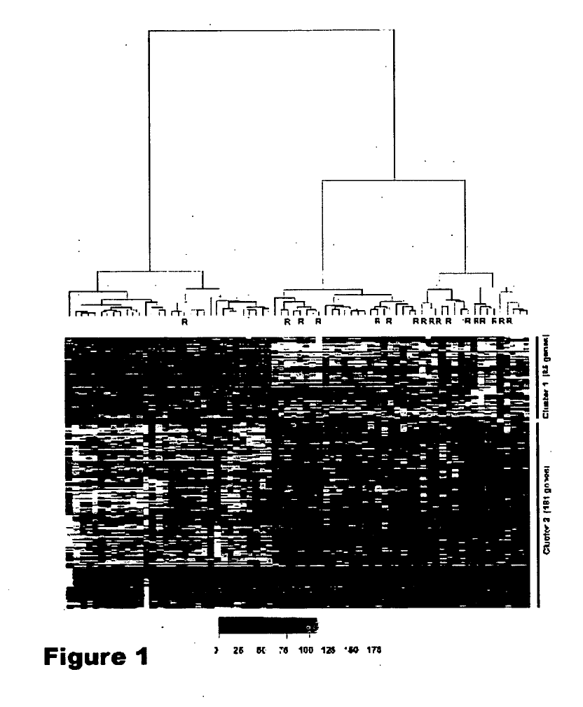

Cluster analysis performed in both dimensions on the 72 RP samples and these 277 probesets using the Ward's minimum variance procedure identified two gene expression subgroups (FIG. 1). Sixteen of the 17 patients known to have experienced a PSA relapse were clustered in one gene expression group characterized by a relative increase in expression of 85 genes (cluster 1) and loss of expression of 179 genes (cluster 2; FIG. 1). An additional 22 patients that were disease-free at the time of censoring were located in this expression cluster, and may suggest that these patients have an increased propensity for relapse in the future. Thirty-two patients who were disease-free at the time of censoring constituted the second expression group which also included one patient who had experienced a PSA relapse.

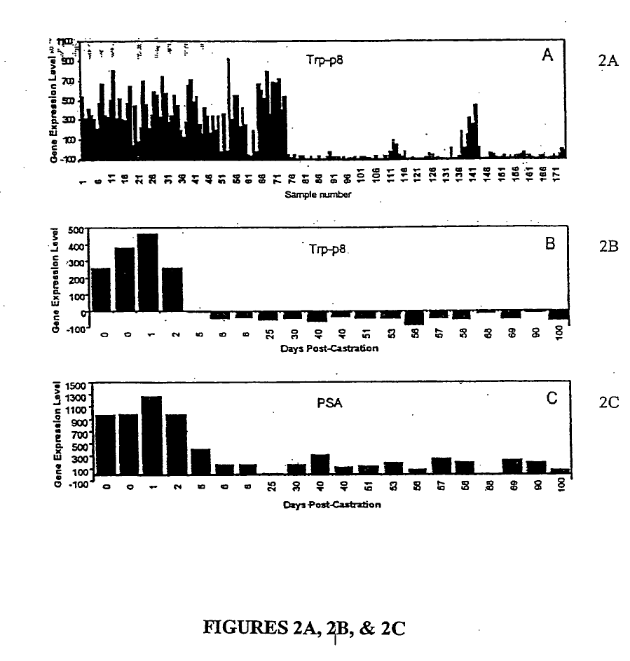

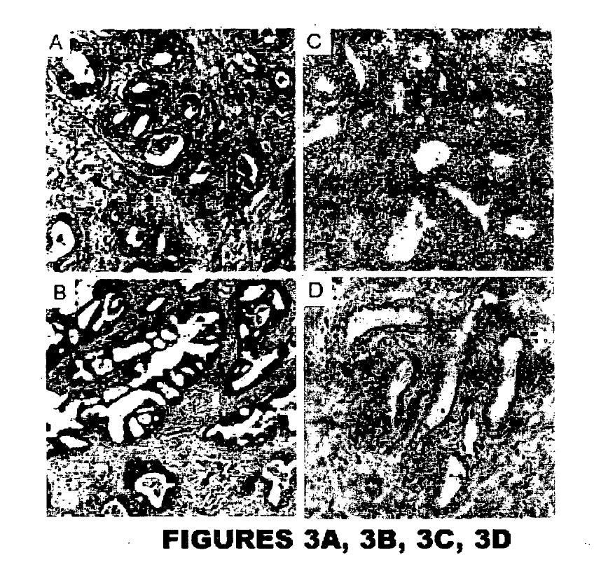

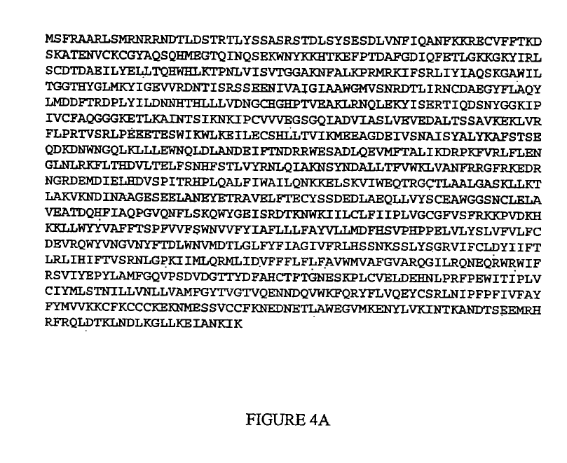

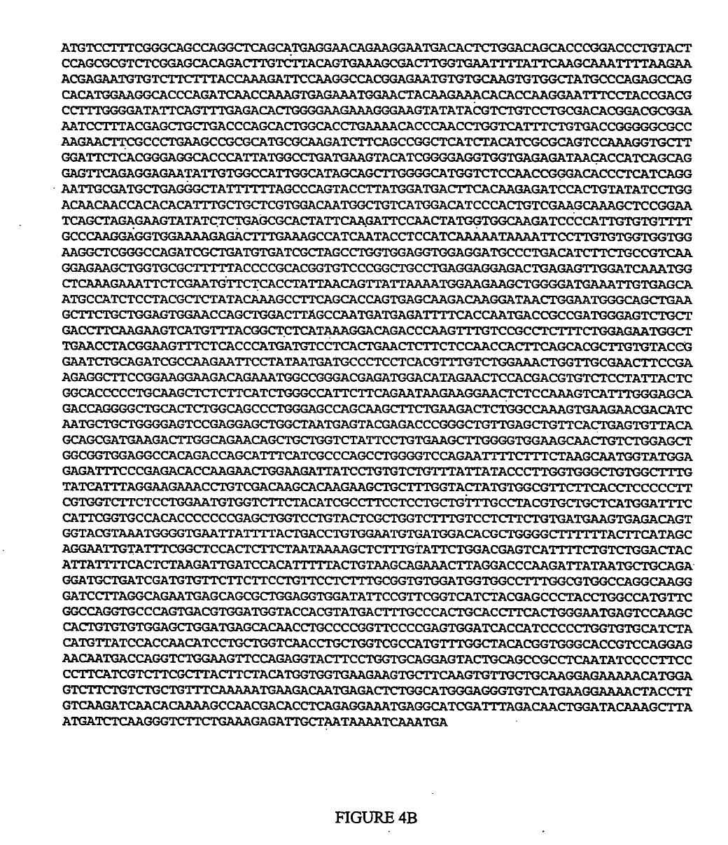

Notably, three of the 277 probesets showing strongest correlation with relapse in our model were identified as the gene for the putative calcium channel protein, trp-p8 (16). For all three probesets, loss of expression of trp-p8 mRNA was associated with a significantly shorter time to PSA relapse free survival with an IQR HR of 0.26 (0.12-0.54; P<0.001), 0.32 (0.16-0.66, P=0.0022) and 0.27 (0.12-0.66, P=0.0045), respectively, when PSA was included in the analysis. Notably, loss of trp-p8 remained a significant predictor of PSA relapse when modeled alone or with Gleason score (data not shown). Subsequent analysis showed that expression of trp-p8 mRNA was primarily restricted to the prostate. Low-level expression was detected in normal liver and no detectable expression was seen in 32 distinct other normal tissues examined by oligonucleotide microarray analysis (FIG. 2a). These data confirm the findings of a recent study that also showed that trp-p8 expression was prostate-specific (16). Analysis of 23 cancer cell lines showed that trp-p8 is only expressed at very low levels in the androgen-dependent prostate cancer cell line LnCaP, but not in the androgen independent prostate cancer cell lines, PC-3 and DU-145, consistent with previous data (16). Since this observation alone is not conclusive evidence that trp-p8 expression is androgen-regulated, we next utilized the androgen-dependent LuCaP-35 prostate cancer xenograft model to assess changes in trp-p8 expression that occur during transition from androgen dependence to androgen independence of prostate cancer (7). Male LuCaP-35 mice were castrated and tumors were harvested at several time points (0-100 days) after castration. High levels of trp-p8 expression were detected on days 0-2 after castration, but not on days 5-100 post castration, and correlated significantly with PSA expression in the same mice (Pearson P=0.080; FIG. 2, B and C).

To gain further insight into the putative association of trp-p8 with androgen regulation, we examined the levels of trp-p8 expression in the prostate tissue of patients who were treated with androgen deprivation therapy (neoadjuvant hormonal therapy, NHT) prior to RP. In situ hybridization (ISH) for trp-8 mRNA was performed on RP specimens from 13 patients who had received at least 3 months preoperative NHT and the levels compared with tissue from 95 patients treated with RP alone (FIG. 3). These latter patients formed part of a large RP cohort described previously (13). While trp-p8 mRNA was detected in 80 of 95 (84%) prostate cancers from patients treated with RP alone, those patients who underwent NHT prior to RP demonstrated significantly less expression of trp-p8, with only 4 of 13 (31%) samples positive for trp-p8 mRNA (Fisher's Exact test, P<0.001; FIG. 3).

Taken together, these data from cell lines, prostate cancer xenografts and clinical specimens, combined with the original finding that trp-p8 mRNA levels correlated strongly with prostate cancer relapse, strongly support the conclusion that trp-p8 expression is androgen-regulated and may be associated with the transition to androgen-independent disease. A monoclonal antibody to trp-p8 can be produced that will be used to assess protein expression by immunohistochemistry in an independent cohort of formalin-fixed, paraffin-embedded prostate cancer specimens with known prostate cancer outcome (13).

### REFERENCES

1. Jemal, A, Thomas, A, Murray, T, Thum, M. Cancer Statistics, 2002. CA Cancer J Clin, 52: 23-47, 2002.
2. Snow, P B, Smith, D S, Catalona, W J. Artificial neural networks in the diagnosis and prognosis of prostate cancer: a pilot study. J Urol, 152: 1923-1926, 1994.
3. Kattan, M W, Eastham, J A, Stapleton, A M, Wheeler, T M, Scardino, P T. A preoperative nomogram for disease recurrence following radical prostatectomy for prostate cancer. J Natl Cancer Instit, 90: 766-771, 1998.
4. D'Amico, A V, Whittington, R, Malkowicz, S B, Fondurulia, J, Chen, M-H, Kaplan, I, Beard, C J, Tomaszewski, J E, Renshaw, A A, Wein, A, Coleman, C N. Pretreatment nomogram for prostate-specific antigen recurrence after radical prostatectomy or external-beam radiation therapy for clinically localised prostate cancer. J Clin Oncol, 17: 168-172, 1999.
5. Graefen, M, Noldus, J, Pichlmeier, A, Haese, P, Hammerer, S, Fernandez, S, Conrad, R, Henke, E, Huland, E, Huland, H. Early prostate-specific antigen relapse after radical retropubic prostatectomy: prediction on the basis of preoperative and postoperative tumor characteristics. Eur Urol, 36: 21-30, 1999.
6. Kattan, M W, Wheeler, T M, Scardino, P T. Postoperative nomogram for disease recurrence after radical prostatectomy for prostate cancer. J Clin Oncol, 17: 1499-1507, 1999.
7. Linja, M J, Savinainen, K J, Saramaki, O R, Tammela, T L J, Vessella, R L, Visakorpi, T. Amplification and overexpression of androgen receptor gene in hormone-refractory prostate cancer. Cancer Res, 61: 3550-3555, 2001.
8. Tukey, J W. Exploratory Data Analysis. Reading, Mass.: Addison-Wesley, 1977.
9. Cox, D R. Regression models and life tables (life tables). J R Stat Soc, 34: 187-189, 1972.
10. Storey, J D, Tibshirani, R. Estimating false discovery rates under dependence, with applications to DNA microarrays. Technical Report, Department of Statistics, Stanford University, CA, 2001.
11. Ward, J H. Hierarchical grouping to optimize an objective function. J Am Statist Assoc, 58: 236-244, 1963.
12. Kononen, J, Bubendorf, L, Kallioniemi, A, Bårlund, M, Schraml, P, Leighton, S. Torhorst, J, Mihatsch, M J, Sauter, G, Kallioniemi, O-P. Tissue microarrays for high-throughput molecular profiling of tumor specimens. Nat Med, 4: 844-847, 1998.
13. Quinn, D I, Henshall, S M, Haynes, A-M, Brenner, P C, Kooner, R, Golovsky, D, Mathews, J, O'Neil, G F, Turner, J J, Delprado, W, Finlayson, J F, Sutherland, R L, Grygiel, J J, Stricker, P D. Prognostic significance of pathological features in localized prostate cancer treated with radical prostatectomy: implications for staging systems and predictive models. J Clin Oncol, 19: 3692-3705, 2001.
14. Platzer, P, Upender, M B, Wilson, K, Willis, J. Lutterbaugh, J, Nosrati, A, Willson, J K V, Mack, D, Ried, T, Markowitz, S. Silence of chromosomal amplifications in colon cancer. Cancer Res, 62: 1134-1138, 2002.
15. Consortium, IHGS. Initial sequencing and analysis of the human genome. Nature, 409: 860-921, 2001.
16. Tsavaler, L, Shapero, M H, Morkowski, S, Laus, R. Trp-p8, a novel prostate-specific gene, is up-regulated in prostate cancer and other malignancies and shares high homology with transient receptor potential calcium channel proteins. Cancer Res., 61: 3760-3769, 2001.
17. Hamdy, F C. Prognostic and predictive factors in prostate cancer. Cancer Treat Rev, 27: 143-151, 2001.
18. van't Veer, L J, Dai, H, van de Vijver, M J, He, Y D, Hart, A A M, Mao, M, Peterse, H L, van der Kooy, K, Marton, M J, Witteveen, A T, Schreiber, G J, Kerkhoven, R M, Roberts, C, Linsley, P S, Bernards, R, Friend, S H. Gene expression profiling predicts clinical outcome of breast cancer. Nature, 415: 530-536, 2002.
19. Perou, C M, Sfrlie, T, Eisen, M B, van de Rijn, M, Jeffrey, S S, Rees, C A, Pollack, J R, Ross, D T, Johnsen, H, Akslen, L A, Fluge, O, Perganrnenschikov, A, Williams, C, Zhu, S X, Lenning, P E, Borresen-Dale, A-L, Brown, P O, Botstein, D. Molecular portraits of human breast tumours. Nature, 406: 747-752, 2000.
20. Gruvberger, S, Ringner, M, Chen, Y, Panavally, S, Saal, L H, Borg, A, Ferno, M, Peterson, C, Meltzer, P S. Estrogen receptor status in breast cancer is associated with remarkably distinct gene expression patterns. Cancer Res, 61: 5979-5984, 2001.
21. Singh, D, Febbo, P G, Ross, K, Jackson, D G, Manola, J, Ladd, C, Tamayo, P, Renshaw, A, D'Amico, A V, Richie, J P, Lander, E S, Loda, M, Kantoff, P W, Golub, T R, Sellers, W R. Gene expression correlates of clinical prostate cancer behavior. Cancer Cell, 1: 203-209, 2002.
22. Clapham, D E, Runnels, L W, Strubing, C. The trp ion channel family. Nature Rev Neuroscience, 2: 387-396, 2001.
23. Isshiki, S, Akakura, K, Komiya, A, Suzuki, H, Kamiya, N, Ito, H. Chromogranin A concentration as a serum marker to predict prognosis after endocrine therapy for prostate cancer. J Urol, 167: 512-515, 2002.
24. Joseph, S K, Boehning, D, Bokkala, S, Watkins, R, Widjaja, J. Biosynthesis of inositol triphosphate receptors: selective association with the molecular chaperone calnexin. Biochem J, 342: 153-161, 1999.
25. Duncan, L M, Deeds, J, Cronin, F E, Donovan, M, Sober, A J, Kauffman, M, McCarthy, J. Melastatin expression and prognosis in cutaneous malignant melanoma. J Clin Oncol, 19: 568-576, 2001.
26. McKemy, D D, Neuhausser, W N, Julius, D. Identification of a cold receptor reveals a general role for TRP channels in thermosensation. Nature, 416: 52-58, 2002.
27. Peier, A M, Moqrich, A, Hergarden, A C, Reeve, A J, Andersson, D A, Story, G M, Earley, T J, Dragoni, I, McIntyre, P, Bevan, S, Patapoutian, A. A TRP channel that senses cold stimuli and menthol. Cell, 108: 705-715, 2002.

It should be apparent that given the guidance, illustrations and examples provided herein, various alternate embodiments, modifications or manipulations of the present invention would be suggested to a skilled artisan and these are included within the spirit and purview of this application and scope of the expanded claims.

What is claimed is:

1. A method of determining prognosis of prostate cancer from a patient, comprising the steps of:
establishing the threshold value of at least one prognostic gene selected from the group of Table 1A, 1B and 1C,
determining the amount of said prognostic gene from a prostate tissue of a patient,
comparing the amount of said prognostic gene with the established threshold value of said prognostic gene, and
determining the prognostic outcome of the patient.

2. The method according to claim 1, wherein said prognostic gene is trp-p8.

3. The method according to claim 1, wherein the amount of said prognostic gene is determined after amplification of said prognostic gene.

4. The method according to claim 1, further comprising determining prostate-specific antigen.

5. The method of claim 1, wherein said prognosis is determined after a selected therapeutic treatment.

6. The method according to claim 1, wherein said prognosis contributes to selection of a therapeutic strategy.

7. The method according to claim 1, wherein said prognosis contributes to selection of a therapeutic strategy.

8. A method of determining prognosis of prostate cancer from a patient, comprising the steps of:
establishing the threshold value of at least one prognostic gene selected from the group of Table 2,
determining the amount of said prognostic gene from a prostate tissue of a patient,
comparing the amount of said prognostic gene with the established threshold value of said prognostic gene, and
determining the prognostic outcome of the patient.


SEQUENCE LISTING (2 sequences)

SEQ ID NO: 1 - protein, 1104 aa, Homo sapiens
MSFRAARLSMRNRRNDTLDSTRTLYSSASRSTDLSYSESDLVNFIQANFKKRECVFFTKDSKATENVCKCGYAQSQHMEGTQINQSEKWNYKKHTKEFPTDAFGDIQFETLGKKGKYIRLSCDTDAEILYELLTQHWHLKTPNLVISVTGGAKNFALKPRMRKIFSRLIYIAQSKGAWILTGGTHYGLMKYIGEVVRDNTISRSSEENIVAIGIAAWGMVSNRDTLIRNCDAEGYFLAQYLMDDFTRDPLYILDNNHTHLLLVDNGCHGHPTVEAKLRNQLEKYISERTIQDSNYGGKIPIVCFAQGGGKETLKAINTSIKNKIPCVVVEGSGQIADVIASLVEVEDALTSSAVKEKLVRFLPRTVSRLPEEETESWIKWLKEILECSHLLTVIKMEEAGDEIVSNAISYALYKAFSTSEQDKDNWNGQLKLLLEWNQLDLANDEIFTNDRRWESADLQEVMFTALIKDRPKFVRLFLENGLNLRKFLTHDVLTELFSNHFSTLVYRNLQIAKNSYNDALLTFVWKLVANFRRGFRKEDRNGRDEMDIELHDVSPITRHPLQALFIWAILQNKKELSKVIWEQTRGCTLAALGASKLLKTLAKVKNDINAAGESEELANEYETRAVELFTECYSSDEDLAEQLLVYSCEAWGGSNCLELAVEATDQHFIAQPGVQNFLSKQWYGEISRDTKNWKIILCLFIIPLVGCGFVSFRKKPVDKHKKLLWYYVAFFTSPFVVFSWNVVFYIAFLLLFAYVLLMDFHSVPHPPELVLYSLVFVLFCDEVRQWYVNGVNYFTDLWNVMDTLGLFYFIAGIVFRLHSSNKSSLYSGRVIFCLDYIIFTLRLIHIFTVSRNLGPKIIMLQRMLIDVFFFLFLFAVWMVAFGVARQGILRQNEQRWRWIFRSVIYEPYLAMFGQVPSDVDGTTYDFAHCTFTGNESKPLCVELDEHNLPRFPEWITIPLVCIYMLSTNILLVNLLVAMFGYTVGTVQENNDQVWKFQRYFLVQEYCSRLNIPFPFIVFAYFYMVVKKCFKCCCKEKNMESSVCCFKNEDNETLAWEGVMKENYLVKINTKANDTSEEMRHRFRQLDTKLNDLKGLLKEIANKIK

SEQ ID NO: 2 - DNA, 3315 bp, Homo sapiens
atgtcctttcgggcagccaggctcagcatgaggaacagaaggaatgacactctggacagcacccggaccctgtactccagcgcgtctcggagcacagacttgtcttacagtgaaagcgacttggtgaattttattcaagcaaattttaagaaacgagaatgtgtcttctttaccaaagattccaaggccacggagaatgtgtgcaagtgtggctatgcccagagccagcacatggaaggcacccagatcaaccaaagtgagaaatggaactacaagaaacacaccaaggaatttcctaccgacgcctttggggatattcagtttgagacactggggaagaaagggaagtatatacgtctgtcctgcgacacggacgcggaaatcctttacgagctgctgacccagcactggcacctgaaaacacccaacctggtcatttctgtgaccgggggcgccaagaacttcgccctgaagccgcgcatgcgcaagatcttcagccggctcatctacatcgcgcagtccaaaggtgcttggattctcacgggaggcacccattatggcctgatgaagtacatcggggaggtggtgagagataacaccatcagcaggagttcagaggagaatattgtggccattggcatagcagcttggggcatggtctccaaccgggacaccctcatcaggaattgcgatgctgagggctattttttagcccagtaccttatggatgacttcacaagagatccactgtatatcctggacaacaaccacacacatttgctgctcgtggacaatggctgtcatggacatcccactgtcgaagcaaagctccggaatcagctagagaagtatatctctgagcgcactattcaagattccaactatggtggcaagatccccattgtgtgttttgcccaaggaggtggaaaagagactttgaaagccatcaatacctccatcaaaaataaaattccttgtgtggtggtggaaggctcgggccagatcgctgatgtgatcgctagcctggtggaggtggaggatgccctgacatcttctgccgtcaaggagaagctggtgcgctttttaccccgcacggtgtcccggctgcctgaggaggagactgagagttggatcaaatggctcaaagaaattctcgaatgttctcacctattaacagttattaaaatggaagaagctggggatgaaattgtgagcaatgccatctcctacgctctatacaaagccttcagcaccagtgagcaagacaaggataactggaatgggcagctgaagcttctgctggagtggaaccagctggacttagccaatgatgagattttcaccaatgaccgccgatgggagtctgctgaccttcaagaagtcatgtttacggctctcataaaggacagacccaagtttgtccgcctctttctggagaatggcttgaacctacggaagtttctcacccatgatgtcctcactgaactcttctccaaccacttcagcacgcttgtgtaccggaatctgcagatcgccaagaattcctataatgatgccctcctcacgtttgtctggaaactggttgcgaacttccgaagaggcttccggaaggaagacagaaatggccgggacgagatggacatagaactccacgacgtgtctcctattactcggcaccccctgcaagctctcttcatctgggccattcttcagaataagaaggaactctccaaagtcatttgggagcagaccaggggctgcactctggcagccctgggagccagcaagcttctgaagactctggccaaagtgaagaacgacatcaatgctgctggggagtccgaggagctggctaatgagtacgagacccgggctgttgagctgttcactgagtgttacagcagcgatgaagacttggcagaacagctgctggtctattcctgtgaagcttggggtggaagcaactgtctggagctggcggtggaggccacagaccagcatttcatcgcccagcctggggtccagaattttctttctaagcaatggtatggagagatttcccgagacaccaagaactggaagattatcctgtgtctgtttattatacccttggtgggctgtggctttgtatcatttaggaagaaacctgtcgacaagcacaagaagctgctttggtactatgtggcgttcttcacctcccccttcgtggtcttctcctggaatgtggtcttctacatcgccttcctcctgctgtttgcctacgtgctgctcatggatttccattcggtgccacacccccccgagctggtcctgtactcgctggtctttgtcctcttctgtgatgaagtgagacagtggtacgtaaatggggtgaattattttactgacctgtggaatgtgatggacacgctggggcttttttacttcatagcaggaattgtatttcggctccactcttctaataaaagctctttgtattctggacgagtcattttctgtctggactacattattttcactctaagattgatccacatttttactgtaagcagaaacttaggacccaagattataatgctgcagaggatgctgatcgatgtgttcttcttcctgttcctctttgcggtgtggatggtggcctttggcgtggccaggcaagggatccttaggcagaatgagcagcgctggaggtggatattccgttcggtcatctacgagccctacctggccatgttcggccaggtgcccagtgacgtggatggtaccacgtatgactttgcccactgcaccttcactgggaatgagtccaagccactgtgtgtggagctggatgagcacaacctgccccggttccccgagtggatcaccatccccctggtgtgcatctacatgttatccaccaacatcctgctggtcaacctgctggtcgccatgtttggctacacggtgggcaccgtccaggagaacaatgaccaggtctggaagttccagaggtacttcctggtgcaggagtactgcagccgcctcaatatccccttccccttcatcgtcttcgcttacttctacatggtggtgaagaagtgcttcaagtgttgctgcaaggagaaaaacatggagtcttctgtctgctgtttcaaaaatgaagacaatgagactctggcatgggagggtgtcatgaaggaaaactaccttgtcaagatcaacacaaaagccaacgacacctcagaggaaatgaggcatcgatttagacaactggatacaaagcttaatgatctcaagggtcttctgaaagagattgctaataaaatcaaatga